SEQUENCE LISTING (4 sequences)

SEQ ID NO: 1 - protein, 12 aa, Artificial sequence
  TUF1 polypeptide
AYKSYVRALPLL

SEQ ID NO: 2 - protein, 6 aa, Artificial sequence
  TUF1 polypeptide
SYVRAL

SEQ ID NO: 3 - protein, 17 aa, Artifical sequence
  TUF1 polypeptide
GTIKLTPRLSKDAYSEM

SEQ ID NO: 4 - protein, 24 aa, Artifical sequence
  TUF1 polypeptide
PLPGNAEEQPSLYEKAPQGKVKVS

(19) United States
(12) Patent Application Publication
(10) Pub. No.: US 2012/0269815 A1
(43) Pub. Date: Oct. 25, 2012

(54) NEUROTROPHINS AND USES THEREOF

(75) Inventor: Phu V. Tran, Minneapolis, MN (US)

(73) Assignee: Regents of the University of Minnesota, St Paul, MN (US)

(21) Appl. No.: 13/141,526

(22) PCT Filed: Dec. 23, 2009
(86) PCT No.: PCT/US09/69454

§ 371 Date: Jun. 22, 2011

Related U.S. Application Data
(60) Provisional application No. 61/203,439, filed on Dec. 23, 2008.

(51) Int. Cl. A61K 38/10; G01N 33/566; G01N 21/64; C07K 7/08; C07K 7/06; C07H 21/00; A61K 31/7088; C07K 16/22; A61K 39/395; C07H 21/02; A61P 25/28; A61P 3/04; A61P 25/16; A61P 9/12; A61P 25/00; A61P 25/22; A61P 15/08; A61K 38/08; C12Q 1/68

(52) U.S. Cl. 424/139.1; 435/6.11; 435/7.1; 530/327; 530/329; 514/8.3; 536/23.5; 514/44 R; 530/387.9; 536/24.5; 530/389.2; 530/388.24

(57) ABSTRACT

A neurotrophin polypeptide, and a biologically active fragment thereof, are described. Also, methods are disclosed that include providing a composition comprising the neurotrophin polypeptide to a subject, wherein the composition is effective to ameliorate at least one symptom or clinical sign of a condition treatable with a neurotrophin when the composition is administered to a subject in need of treatment for a condition treatable with a neurotrophin.

CONTINUING APPLICATION DATA

This application claims the benefit of U.S. Provisional Application Ser. No. 61/203,439, filed Dec. 23, 2008, which is incorporated by reference herein.

### BACKGROUND

Epidemiological studies of human populations suggest that exposure to adverse environmental conditions (e.g., stress and/or poor nutrition) during gestational and neonatal periods can have deleterious influences on the development of physiology (e.g., autonomic nervous system, neuroendocrine system, immune function, etc.), cognitive function, and behavior. These influences can persist into adulthood, manifested as increased risk for developing certain physiological and/or neurologic disorders. Studies performed in animal models confirm acute and long-term effects of exposure to adverse environmental conditions during gestational and/or neonatal periods.

The limbic (Amygdala, cortex and hippocampus) Hypothalamic Pituitary Adrenal (LHPA) axis is a neuroendocrine circuit that mediates a wide range of behavioral and physiological activity critical for survival such as, for example, reproductive and parenting behaviors, cognitive function, fight or flight responses, awake/sleep (arousal) states, and energy metabolism. For example, certain psychological (e.g., perceived threat, anxiety, etc.), physical (e.g., pain, restraint, etc.), or physiological (e.g., hemorrhage, exposure to temperature extremes, etc.) stimuli activate the release of corticotrophin releasing factor (CRF) and arginine vasopressin (AVP) from the hypothalamic median eminence into the portal circulation of the hypophysis. CRF and AVP can stimulate corticotrophs in the pituitary to secrete adrenocorticotropic hormone (ACTH) into the general circulation. ACTH, in turn, can induce glucocorticoid (GC) secretion from the adrenal cortex. Elevated GC can activate GC receptors in the brain and pituitary to cease the release of CRF and ACTH, thereby restoring the basal (e.g., homeostatic) state. GC also can act on target tissues to induce physiological responses such as, for example, increased blood pressure, decreased appetite, mobilized immune responses, and directing energy metabolism to the brain and musculature.

Adverse early-life experiences can alter the developmental programming of the LHPA circuit. However, factors responsible for these programming effects remain largely unknown. While the long-term effects of early-life adversities can be explained in part by GC receptor-mediated genomic changes, it is possible that other contributing neuropeptide(s) or hormone(s) has yet to be identified. Such factors may mediate the long-term effects of early-life adversity independently of the CRF/GC/GR system.

### SUMMARY OF THE INVENTION

Neurotrophins regulate important aspects of neural development and function (e.g., differentiation, survival and plasticity), which influences the developmental biology of the brain, neurodegenerative diseases etiology, and psychiatric disorders manifestation. TUF1 appears to be a novel neurotrophin, which has a similar effect as other family members, and also may have a role in, regulating stress responses, hypertension, and energy balance.

Accordingly, in one aspect, the present invention provides a TUF1 polypeptide. A TUF1 polypeptide may include the amino acid sequence depicted in SEQ ID NO:1. In other embodiments, the TUF1 polypeptide may include the amino acid sequence depicted in SEQ ID NO:2.

In another aspect, the present invention can include a polynucleotide that encodes a TUF1 polypeptide. The polynucleotide may encode the amino acid sequence depicted in SEQ ID NO:1. In other embodiments, the polynucleotide may encode the amino acid sequence depicted in SEQ ID NO:2.

In another aspect, the present invention provides antibodies that specifically bind to at least a portion of a TUF1 polypeptide. In some embodiments, the antibody may specifically bind to at least a portion of the amino acid sequence depicted in SEQ ID NO:1. In other embodiments, the antibody may specifically bind to at least a portion of the amino acid sequence depicted in SEQ ID NO:2.

In another aspect, the present invention provides a method that includes, generally, providing a composition that includes a TUF1. In certain embodiments, the composition that includes a TUF1 polypeptide can be effective to ameliorate at least one symptom or clinical sign of a condition treatable with a neurotrophin. Consequently, a TUF1 polypeptide may be a prophylactic and/or therapeutic option for therapies that involve administration of a neurotrophin. In some embodiments, the TUF1 polypeptide may include the amino acid sequence depicted in SEQ ID NO:1. In other embodiments, the TUF1 polypeptide may include the amino acid sequence depicted in SEQ ID NO:2. The composition may be effective for use in therapies for condition resulting from neuron trauma such as, for example, stroke, hypothermia, hypoxia, and hyponutrition. The composition may also be effective for use in therapies for conditions such as, for example, hypertension, eating disorders, phobias/anxiety, and neurological disorders.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1 depicts the amino acid sequence of SEQ ID NO:1.

FIG. 2 depicts the amino acid sequence of SEQ ID NO:2.

FIG. 3 shows in situ hybridization showing tuf1 expression in the developing CNS of mouse embryo. In situ hybridization showing tuf1 expression in the developing CNS of gestational day 11.5 mouse embryo (Panel 1, saggital section), gestational day 15.5 (Panel 2, coronal section), neonatal brain (Panel 3, coronal section), and the adult mouse brain (Panels 4-8). Panel 4 shows the sense control. A—amygdala, Arc—Arcuate nucleus of hypothalamus, Ccx—cingulate cortex, DG—dentate gyrus of the hippocampus, Dmh—dorsomedial hypothalamus, fb—forebrain region, H—hippocampus, h—hypothalamic region, Hb—Habenula, hb—hindbrain region, mb—midbrain region, Ncx—neocortex, OG—otic ganglion, p—pituitary, Pcx—piriform cortex, S—spinal cord, TG—trigerminal ganglion, Vmh—ventromedial hypothalamus.

FIG. 4 shows expression of TUF1 polypeptide in postnatal day 15 rat. Expression of TUF1 protein in postnatal day 15 rat hippocampus (Panels A-D), layer III and VI of the neocortex (Panel F), piriform cortex (Panel F), basomedial amygdale (Panel G), ventromedial nucleus of the hypothalamus (Panel H), and habenula (Panels I-L). TUF1 (Green) is expressed primarily in neurons (NeuN-Red) (Panel J, an expansion of dotted square in panel I) and also in oligodendrocytes (Myelin-Red, panel L, an enlargement of dotted square in Panel K) as shown for Habenula. Unmark white scale bar=100 μm.

FIG. 5 shows TUF1 polypeptide expression in adult mouse. TUFT expression in adult mouse pituitary and adrenal. In the pituitary, bottom panels show schematic sketches of cells expressing TUF1 as found in top panels. Dashline represents plane of section across the pituitary. In the adrenal, TUF1 is found in the zona glomerulosa and is overlapped with the P450 aldosterone synthase (bottom panels). ZG—zona glomerulosa, ZF—zona fasciulata, ZR—zona reticulate.

FIG. 6 shows Na⁺-restriction induced expansion of the P450 aldosterone synthase, nerve terminals, and TUF1 polypeptide expression. Na-restriction induced expansion of the P450 aldosterone synthase (AS), nerve terminals as shown by the expression of NPY and p75^{NTR}, and TUF1 expression. Na-replacement (bottom panels) following restriction reduced AS expression, neurite outgrowth, and TUF1 expression. Data were quantified for each diet condition (insets). Statistical calculations were performed using Graphpad Prism, Student's t-test, n=4/group, *p<0.05, **p<0.01, ***p<0.001.

FIG. 7 shows conserved elements of the regulatory region of tuf1. Conserved elements in the regulatory region of tuf1 gene (I, II and III). Sequence comparison of 10 Kb upstream of tuf1 transcription start site among mouse, rat, and human DNA using Vista software (http://genome.lbl.gov/vista/index.shtml).

FIG. 8 shows membrane-bound vesicle localization and post-translational cleavage of TUF1 polypeptide. Membrane-bound vesicle localization and post-translational cleavage of TUF1 protein. COST cells expressed Red-fluorescence protein (Panel A, RFP) or C-terminal TUF1 tagged-RFP (Panel B). Tagged protein is confined to vesicles, and not cortical membrane. TUF1 protein was detected along the axon ov VMH neurons (Panel C, arrows). Western blot analysis of adrenal proteins revealed two TUF1 products (Panel D, asterisks), suggesting a full-length and a cleaved product. Panel E illustrates TUF1 protein with C-terminus targeted by the antisera (Red line).

FIG. 9 shows TUF1 binding by p75^{NTR} expressed by COS cells. TUF1 peptide binds p75 receptor expressed in COS cells (left panel, arrowheads) and an unidentified receptor (arrow). Proposed model for TUF1 as a modulator of neuronal activity in promoting growth, differentiation and survival.

FIG. 10 shows diurnal expression of tuf1 mRNA in rat. Diurnal expression of tuf1 mRNA in rat hypothalamus and adrenal. (n=4/group, *p<0.001, ANOVA).

FIG. 11 shows tuf1 mRNA in the VMH of mice exposed to acute cold stress. tuf1 mRNA in the VMH of mice exposed to acute cold stress (Panel A) or overnight food-deprivation (Panel B). Dissected VMH from 4 mice per stressor were combined and analyzed for vesiculated glutamate transporter 2 (Vglut2) and tuf1 mRNAs. (Student's t-test, *p<0.01).

FIG. 12 shows tuf1 mRNA expression in rat given iron deficient (ID) diet. Quantitative measurement of mRNA in the rat hippocampus. (A) tuf1 mRNA level (2-way ANOVA, n=6/group/postnatal age, p=0.024 for iron status, and p<0.0001 for developmental curve). (B) Glucocorticoid receptor (GR) mRNA. GR expression peaks at P30. Both control (IS, square, solid line) and perinatal iron deficiency (ID, circle, dashed line) showed similar developmental trajectory. Acute increase of GR level occurred in P30 ID rats. (Bonferroni posthoc t-test, n=6/group/postnatal day, **p<0.01).

FIG. 13 shows an in vitro assessment of TUF[43-54] binding to p75^{NTR}. In vitro assessment of TUF1[43-54] binding to p75^{NTR}. (A-C) COS7 cells transfected with pCMV-GFP and incubated with TUF1[43-54]. (D-F) COS7 cells transfected with pCMV-SPORT6-p75 and incubated with TUF1[43-54]. (G-I) COS7 cells transfected with pCMV-SPORT6-p75 and incubated with TUF1[24-40]. Cells were counterstained with DAPI (blue).

FIG. 14 shows TUF1 expression in adrenal glomerulosa in rats provided with a sodium-restricted diet.

FIG. 15 shows TUF1 expression in adult rat adrenal. TUF1 expression in adult rat adrenal. A. TUF1 is expressed primarily in the adrenal cortex. B. Enlarged image of Panel A (dashed box) showing TUF1 expression in glomerulosa zone. Scale bar=50 μm.

FIG. 16 shows an in vitro assessment of TUF1[43-54] binding to p75^{NTR}.

FIG. 17 shows sodium replacement results in regression of zona glomerulosa.

FIG. 18 shows the effects of sodium diet manipulations on expression of aldosterone synthase, NPY-immunoreactive (ir) neurites, p75^{NTR}-ir neurites, and TUF1[153-167] polypeptide. Control vs. Na⁺ restriction/replacement graphs. Sodium diet manipulations altered expression of aldosterone synthase (A), NPY+ neuritis (B), p75NTR+ neuritis (C), and TUF1 (D) in rat adrenal. Values are mean±SEM, n=4, Student's t-test; *p,0.05, **p<0.01, ***p<0.001.

FIG. 19 shows TUF1[43-54] reduces in vitro cell death following serum deprivation. (A) A micrograph of COST cells stained with Trypan blue. (B) Graphs of Trypan blue-positive (+) cells counted at 24, 48 and 72 hours of serum deprivation. Values are means±SEM, n=3. *Asterisk denotes P<0.05, t-test with Welch's correction.

FIG. 20 shows activated-Caspase 3 expression in primary cultured hypothalamic neurons.

FIG. 21 depicts the amino acid sequence of SEQ ID NO:3.

FIG. 22 depicts the amino acid sequence of SEQ ID NO:4.

FIG. 23 shows that Angiotensin II (ANG II) activates TUF1 expression in adrenal glomerulosa cell dispersion.

FIG. 24 shows that suppression of TUF1 mRNA (left panel) or TUF1[43-54] (right panel) reduces GT1-1 cell survival following serum deprivation. GT1-1 cells were transfected with RNAi targeting TUF1 mRNA or treated with anti-TUF1 IgG immediately following serum deprivation. After 48-hr of serum deprivation, both treatments showed fewer surviving cells compared to the non-treated control group.

FIG. 25 shows that TUF1[43-54] peptide promotes GT1-1 cell survival following serum deprivation. GT1-1 cells are immortalized hypothalamic neural precursors (a gift from Dr. Richard Wiener, UCSF). Following 24 hr of serum deprivation, 70% of cells survived with TUF1 peptide (1.8 μM) supplementation compared to 48% of cells survived without supplementation. In contrast, TUF1 peptide has no effect at 3.6 μM concentration. Similar survival effect of a lower TUF1 dose was also observed for 48 hr. post serum deprivation. These data demonstrates that TUF1 can confer neurotrophic activity at a lower dose (1.8 μM), whereas it might have cytotoxic effect at a higher dose (3.6 μM).

FIG. 26 shows that RNAi suppression of TUF1 impairs retinoic acid-induced GnRH expression in GT1-1 cells. GT1-1 cell expresses TUF1, which can be suppressed by RNAi. Three days following transfection with RNAi, cells were stimulated with retinoic acid to induce gonadotropin releasing hormone (GnRH) expression, marking the differentiation of neural precursors into GnRH neurons. GT1-1 cells transfected with RNAi targeting TUF1 transcript reduced 69% TUF1 mRNA compared to a negative control RNAi and produced 79% less GnRH mRNA. These data suggest TUF1 mediates aspects of neuronal differentiation.

FIG. 27 shows that TUF1[43-54] peptide reduces neural excitotoxicity induced by glutamate.

FIG. 28 shows that TUF1 expression is activated in the cortex in a Huntington's Disease mouse model.

FIG. 29 shows that early-life nutrient deficiencies suppress TUF1 expression in the limbic system. Hypoglycemia and iron deficiency are common nutrient deficiencies during early-life, affecting brain development with long-term sequelae. Both conditions reduced 30-40% TUF1 mRNA in the rat hippocampus and cortex. Moreover, adult rats that were iron-deficient only during the gestational-neonatal period had 40% lower hippocampal TUF1 mRNA compared with always iron sufficient controls, suggesting a long-term effect in TUF1 regulation.

FIG. 30 shows that administration of TUF1[43-54] influences food consumption in a rat model.

FIG. 31 shows that administration of TUF1[43-54] alters contextual fear response in a rat model. Adult male rats administered TUF1 peptide (1.0 μg or 10 μg) via an indwelling cannulae implanted into the lateral ventricle showed reduced fear potentiated startle than control subjects, suggesting TUF1 is involved in regulating contextual fear response. No effect on shock reactivity, shock sensitization or pre-pulse inhibition suggest TUF1 affects specifically fear potentiated response independent of pain sensitivity or sensorimotor gating.

FIG. 32 depicts TUF1 expression in fat tissues.

FIG. 33 depicts TUF1 expression in Leydig cells.

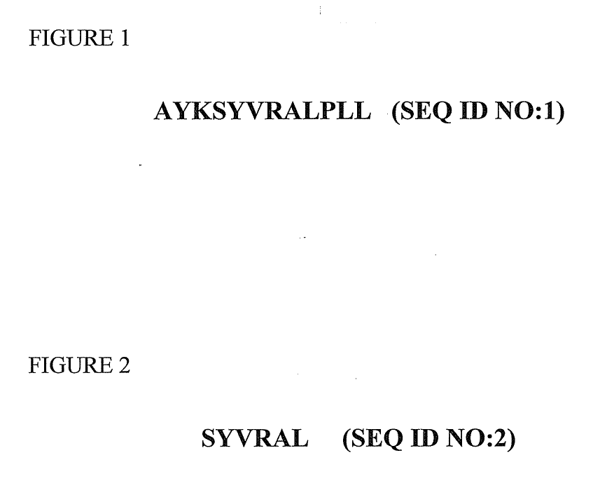

### DETAILED DESCRIPTION OF ILLUSTRATIVE EMBODIMENTS

Early-life adversities have maladaptive impacts on the developmental trajectory of brain and endocrine systems critical for cognitive function, emotional behavior, and physiology. Chronic infections, nutritional deficiencies, and poor maternal/infant interaction result in impaired growth and abnormal neurologic and physiologic development. The limbic-hypothalamic pituitary adrenal (LHPA) circuitry plays a central role in orchestrating physiological functions vital for survival. Yet, relatively few molecules have been identified that responsible for the environmental response of this circuitry. Furthermore, little is known about how adverse environmental stimuli are translated to signals in the limbic system and integrated into a coordinate response in the HPA axis.

Neurotrophins are family of relatively small polypeptides and receptors that regulate neuronal development and maintenance. Neurotrophins are secreted by target tissue as well as neuron and act by preventing the associated neuron from initiating programmed cell death, thereby allowing the neurons to survive and thrive. Neurotrophins also promote proliferation and differentiation of progenitor cells to form neurons.

Neurotrophins therefore play critical roles in neuronal differentiation, survival and plasticity, which influence the developmental biology of the brain, neurodegenerative diseases, and psychiatric disorders. The present invention relates to a novel neurotrophin, referred to herein as TUF1, that exhibits activities similar to other neurotrophins, but also may have a role in regulating stress responses, cognitive and emotional behaviors, hypertension, feeding behavior, and energy mobilization. Consequently, the TUF1 polypeptide may be useful for therapies used to treat, for example, hypertension, neuromodulation, stress, hormone dysregulation, eating disorders, phobias, anxiety, and neurological disorders.

TUF1 is an evolutionary conserved novel polypeptide with a potentially secreted motif that is highly homologous to the p75 receptor-binding domain of neurotrophic factors. Indeed, this motif of TUF1 was demonstrated to bind the p75 receptor. TUF1 is likely a secreted neuropeptide based on its localization in membrane-bound vesicles. Moreover, the gene encoding TUF1, tuf1, is expressed in the neuroendocrine circuitry including, for example, the cortex, amygdala, hippocampus, hypothalamus, pituitary, and adrenal cortex. Also, expression of tuf1 is regulated by acute and chronic stressors such as, for example, exposure to extreme temperatures, food deprivation, hypoglycemia, and iron deficiency. Taken together, these findings implicate TUF1 involvement in mediating physiological homeostasis, cognitive function, and emotional behavior.

### Definitions

“Ameliorate” refers to any reduction in the extent, severity, frequency, and/or likelihood of a symptom or clinical sign characteristic of a particular condition.

“HPA axis” refers, collectively, to the hypothalamus, pituitary, and adrenal gland.

“Limbic axis” refers, collectively, to the hippocampus, cortex, and amygdala

“Limbic-HPA axis” refers, collectively, to the limbic axis and HPA axis.

“Neurotrophin” refers to a molecule (e.g., a polypeptide) that promotes the survival and plasticity of neurons. A neurotrophin may induce differentiation of a progenitor cell to form a neuron.

“Prophylactic” and variations thereof refer to a treatment that limits, to any extent, the development and/or appearance of a symptom or clinical sign of a condition.

“Sign” or “clinical sign” refers to an objective physical finding relating to a particular condition capable of being found by one other than the patient.

“Symptom” refers to any subjective evidence of disease or of a patient's condition.

“Therapeutic” and variations thereof refer to a treatment that ameliorates one or more existing symptoms or clinical signs associated with a condition.

“Treat” or “treatment” or any variation thereof refers to reducing, ameliorating, or resolving, to any extent, the symptoms or signs related to a condition.

The term “and/or” means one or all of the listed elements or a combination of any two or more of the listed elements.

The terms “comprises” and variations thereof do not have a limiting meaning where these terms appear in the description and claims.

Unless otherwise specified, “a,” “an,” “the,” and “at least one” are used interchangeably and mean one or more than one. For example, a composition that includes “a” polypeptide can encompass a composition that includes a single polypeptide as well as a composition that includes one or more polypeptides.

Also herein, the recitations of numerical ranges by endpoints include all numbers subsumed within that range (e.g., 1 to 5 includes 1, 1.5, 2, 2.75, 3, 3.80, 4, 5, etc.).

In one aspect, the present invention provides polypeptide comprising a TUF1 polypeptide. As used herein, “polypeptide” refers to a polymer of amino acids linked by peptide bonds. Thus, for example, the terms peptide, oligopeptide, protein, and enzyme are included within the definition of polypeptide. This term also includes post-expression modifications of the polypeptide, such as glycosylations, acetylations, phosphorylations, and the like. The term polypeptide does not connote a specific length of a polymer of amino acids. A polypeptide may be isolatable directly from a natural source, or can be prepared with the aid of recombinant, enzymatic, or chemical techniques. In the case of a polypeptide that is naturally occurring, such a polypeptide is typically isolated. An “isolated” polypeptide is one that has been removed from its natural environment. For instance, an isolated polypeptide is a polypeptide that has been removed from the cytoplasm or from the membrane of a cell, and many of the polypeptides, nucleic acids, and other cellular material of its natural environment are no longer present. An “isolatable” polypeptide is a polypeptide that could be isolated from a particular source. A “purified” polypeptide is one that is at least 60% free, for example at least 75% free, for example at least 90% free from other components with which they are naturally associated. Polypeptides that are produced outside the organism in which they naturally occur, e.g., through chemical or recombinant means, are considered to be isolated and purified by definition, since they were never present in a natural environment. As used herein, a “polypeptide fragment” refers to a portion of a polypeptide that results from digestion of a polypeptide with a protease.

As used herein, a “TUF1 polypeptide” demonstrates one or more of the functional activities of the twelve amino acid sequence depicted in SEQ ID NO:1. Exemplary functional activities of a TUF1 polypeptide, and assays for measuring these functional activities, are described in more detail herein. Briefly, functional activities of a TUF1 polypeptide include, but are not limited to, one or more of the following: specific binding with the p75 neurotrophic receptor, promoting COS7 cell survival, promoting neural cell survival, mediating amygdala-based fear responses, and mediating stress-induced drug-seeking behavior.

The TUF1 polypeptides of the present invention may be derived from a variety of species of mammals including, but not limited to, humans, primates, rats, mice, cows, pigs, dogs, etc.

The polypeptides of the present invention also include “biologically active analogs” of naturally occurring polypeptides. As used herein, a “biologically active analog” demonstrates one or more of the following functional activities: specific binding with the p75 neurotrophic receptor, promoting COS7 cell survival, promoting neural cell survival, mediating amygdala-based fear responses, mediating stress-induced drug-seeking behavior, and/or any of the activities demonstrated in Example 1 through Example 14, below. Functional activity of a TUF1 polypeptide can be assessed using the various assays described herein as well as other assays well known to one with ordinary skill in the art. A modulation in functional activity, including the stimulation or the inhibition of functional activity, can be readily ascertained by the various assays described herein, and by assays known to one of skill in the art.

A modulation in a functional activity can be quantitatively measured and described as a percentage of the functional activity of a comparable control. The functional activity of the present invention includes a modulation that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 110%, at least 125%, at least 150%, at least 200%, or at least 250% of the activity of a suitable control.

For example, the stimulation of a functional activity of a TUF1 polypeptide can be quantitatively measured and described as a percentage of the functional activity of a comparable control. Stimulation of a functional activity of a TUF1 polypeptide includes a stimulation that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 110%, at least 125%, at least 150%, at least 200%, or at least 250% greater than the activity of a suitable control.

For example, inhibition of a functional activity of a TUF1 polypeptide can be quantitatively measured and described as a percentage of the functional activity of a comparable control. Inhibition of a functional activity of a TUF1 polypeptide includes an inhibition that is no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, no more than 90%, no more than 95%, no more than 99%, or no more than 100% of the activity of a suitable control.

A “biologically active analog” of a polypeptide includes polypeptides having one or more amino acid substitutions that do not eliminate a functional activity. Substitutes for an amino acid in the polypeptides of the invention may be selected from other members of the class to which the amino acid belongs. For example, it is well-known in the art of protein biochemistry that an amino acid belonging to a grouping of amino acids having a particular size or characteristic (such as charge, hydrophobicity and hydrophilicity) can be substituted for another amino acid without altering the activity of a protein, particularly in regions of the protein that are not directly associated with biological activity. Substitutes for an amino acid may be selected from other members of the class to which the amino acid belongs. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Examples of such preferred conservative substitutions include Lys for Arg and vice versa to maintain a positive charge; Glu for Asp and vice versa to maintain a negative charge; Ser for Thr so that a free —OH is maintained; and Gln for Asn to maintain a free NH2. Likewise, biologically active analogs of a TUF1 polypeptide containing deletions or additions of one or more contiguous or noncontiguous amino acids that do not eliminate a functional activity of a TUF1 polypeptide are also contemplated.

A “biologically active analog” of a TUF1 polypeptide includes “fragments” and “modifications” of a TUF1 polypeptide. As used herein, a “fragment” of a TUF1 polypeptide means a TUF1 polypeptide that has been truncated at the N-terminus, the C-terminus, or both. A fragment may range from about 4 to about 8 amino acids in length. For example it may be 4, 5, 6, 7, or 8 amino acids in length. Fragments of a TUF1 polypeptide with potential biological activity can be identified by many means. One means of identifying such fragments of a TUF1 polypeptide with biological activity is to compare the amino acid sequences of a TUF1 polypeptide from rat, mouse, human and/or other species to one another. Regions of homology can then be prepared as fragments. Fragments of a polypeptide also include a portion of the polypeptide containing deletions or additions of one or more contiguous or noncontiguous amino acids such that the resulting polypeptide still retains a biological activity of the full-length polypeptide. On exemplary fragment of a TUF1 polypeptide is the six amino acid fragment depicted in SEQ ID NO:2.

A “modification” of a TUF1 polypeptide includes TUF1 polypeptides or fragments thereof chemically or enzymatically derivatized at one or more constituent amino acid, including side chain modifications, backbone modifications, and N- and C-terminal modifications including acetylation, hydroxylation, methylation, amidation, and the attachment of carbohydrate or lipid moieties, cofactors, and the like. Modified polypeptides of the invention may retain the biological activity of the unmodified polypeptide or may exhibit a reduced or increased biological activity.

The polypeptides and biologically active analogs thereof of the present invention include native (naturally occurring), recombinant, and chemically or enzymatically synthesized polypeptides. For example, the TUF1 polypeptides of the present invention may be prepared by isolation from naturally occurring tissues or prepared recombinantly, by well known methods, including, for example, preparation as fusion proteins in bacteria and insect cells.

The polypeptides of the present invention include polypeptides with “structural similarity” to the polypeptide depicted in SEQ ID NO:1 and/or the fragment polypeptide depicted in SEQ ID NO:2. As used herein, “structural similarity” refers to the identity between two polypeptides. For polypeptides, structural similarity is generally determined by aligning the residues of the two polypeptides (for example, a candidate polypeptide and the polypeptide of SEQ ID NO:1 or SEQ ID NO:2) to optimize the number of identical amino acids along the lengths of their sequences; gaps in either or both sequences are permitted in making the alignment in order to optimize the number of identical amino acids, although the amino acids in each sequence must nonetheless remain in their proper order. A candidate polypeptide is the polypeptide being compared to the polypeptide of SEQ ID NO:1 or SEQ ID NO:2. A candidate polypeptide can be isolated, for example, from an animal, or can be produced using recombinant techniques, or chemically or enzymatically synthesized.

A pair-wise comparison analysis of transporter protein sequences can carried out using the BESTFIT algorithm in the GCG package (version 10.2, Madison, Wis.). Alternatively, polypeptides may be compared using the Blastp program of the BLAST 2 search algorithm, as described by Tatiana et al., (FEMS Microbiol Lett, 174, 247-250 (1999)), and available on the world wide web at ncbi.nlm.nih.gov/BLAST/. The default values for all BLAST 2 search parameters may be used, including matrix=BLOSUM62; open gap penalty=11, extension gap penalty=1, gap x_dropoff=50, expect=10, wordsize=3, and filter on.

In the comparison of two amino acid sequences, structural similarity may be referred to by percent “identity” or may be referred to by percent “similarity.” “Identity” refers to the presence of identical amino acids and “similarity” refers to the presence of not only identical amino acids but also the presence of conservative substitutions.

A TUF1 polypeptide of the present invention include polypeptides with at least 50%, at least 58%, at least 66%, at least 75%, at least 83%, or at least 91% sequence similarity to the amino acid sequence of SEQ ID NO:1. A TUF1 polypeptide of the present invention also includes polypeptides with at least 50%, at least 66%, or at least 83% sequence similarity to the amino acid sequence of SEQ ID NO:2.

A TUF1 polypeptide of the present invention include polypeptides with at least 50%, at least 58%, at least 66%, at least 75%, at least 83%, or at least 91% sequence identity to the amino acid sequence of SEQ ID NO:1. A TUF1 polypeptide of the present invention also includes polypeptides with at least 50%, at least 66%, or at least 83% sequence identity to the amino acid sequence of SEQ ID NO:2.

A TUF1 polypeptide can include additional amino acids derived from the full-length TUF1 protein such as, for example, the amino acid sequence depicted in SEQ ID NO:3. In some cases, the TUF1 polypeptide can include an amino acid sequence with at least 58%, at least 64%, at least 70%, at least 76%, at least 82%, at least 88%, or at least 94% sequence similarity to the amino acid sequence depicted in SEQ ID NO:3. In other embodiments, the TUF1 polypeptide can include an amino acid sequences with at least 58%, at least 64%, at least 70%, at least 76%, at least 82%, at least 88%, or at least 94% sequence identity to the amino acid sequence depicted in SEQ ID NO:3.

The polypeptides of the present invention can also be designed to provide additional sequences, such as, for example, the addition of coding sequences for added C-terminal or N-terminal amino acids that may facilitate purification by trapping on columns or use of antibodies. Such tags include, for example, histidine-rich tags that allow purification of polypeptides on nickel columns. Such gene modification techniques and suitable additional sequences are well known in the molecular biology arts.

Amino acids essential for the function of TUF1 polypeptides can be identified according to procedures known in the art, such as site-directed mutagenesis or alanine-scanning mutagenesis (Cunningham and Wells, Science 244: 1081-1085, 1989; Bass et al., Proc. Natl. Acad. Sci. USA 88: 4498-4502, 1991).

The polypeptides of the present invention may be formulated in a composition along with a “carrier.” As used herein, “carrier” includes any solvent, dispersion medium, vehicle, coating, diluent, antibacterial and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions.

By “pharmaceutically acceptable” is meant a material that is not biologically or otherwise undesirable, i.e., the material may be administered to an individual along with a TUF1 polypeptide without causing any undesirable biological effects or interacting in a deleterious manner with any of the other components of the pharmaceutical composition in which it is contained.

A TUF1 polypeptide may be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). It is foreseen that a composition can be administered to a mucosal surface, such as by administration to, for example, the nasal or respiratory mucosa (e.g., by spray or aerosol). A composition also can be administered via a sustained or delayed release.

A formulation may be conveniently presented in unit dosage form and may be prepared by methods well known in the art of pharmacy. Methods of preparing a composition with a pharmaceutically acceptable carrier include the step of bringing the TUF1 polypeptide into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

A TUF1 polypeptide may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

The amount of TUF1 polypeptide administered can vary depending on various factors including, but not limited to, the specific TUF1 polypeptide, the weight, physical condition, and/or age of the subject, and/or the route of administration. Thus, the absolute weight of the TUF1 polypeptide included in a given unit dosage form can vary widely, and depends upon factors such as the species, age, weight and physical condition of the subject, as well as the method of administration. Accordingly, it is not practical to set forth generally the amount that constitutes an amount of the TUF1 polypeptide effective for all possible applications. Those of ordinary skill in the art, however, can readily determine the appropriate amount with due consideration of such factors.

In some embodiments, the methods of the present invention include administering sufficient TUF1 polypeptide to provide a dose of, for example, from about 100 ng/kg to about 50 mg/kg to the subject, although in some embodiments the methods may be performed by administering TUF1 polypeptide in a dose outside this range. In some of these embodiments, the method includes administering sufficient TUF1 polypeptide to provide a dose of from about 10 μg/kg to about 5 mg/kg to the subject, for example, a dose of from about 100 μg/kg to about 1 mg/kg.

Alternatively, the dose may be calculated using actual body weight obtained just prior to the beginning of a treatment course. For the dosages calculated in this way, body surface area (m²) is calculated prior to the beginning of the treatment course using the Dubois method: m²=(wt kg^{0.425}×height cm^{0.725})×0.007184.

In some embodiments, the methods of the present invention may include administering sufficient TUF1 polypeptide to provide a dose of, for example, from about 0.01 mg/m² to about 10 mg/m².

In some embodiments, the TUF1 polypeptide may be administered, for example, from a single dose to multiple doses per week, although in some embodiments the methods of the present invention may be performed by administering the TUF1 polypeptide at a frequency outside this range. In certain embodiments, the TUF1 polypeptide may be administered from about once per month to about five times per week.

In another aspect, the present invention is directed to methods for making antibodies, for example, by either inducing the production of antibody in an animal or by recombinant techniques. The antibody produced includes antibody that specifically binds at least one TUF1 polypeptide or fragment thereof. The present invention further includes antibody that specifically binds to a TUF1 polypeptide or fragment thereof, and compositions including such antibodies.

The method may be used to produce an antibody composition that specifically binds a TUF1 polypeptide. As used herein, an antibody that can “specifically bind” a TUF1 polypeptide is an antibody that interacts with the epitope of the TUF1 polypeptide or interacts with a structurally related epitope and/or having a differential or a non-general (i.e., non-specific) affinity, to any degree, for a TUF1 polypeptide. In some embodiments, an antibody composition can include polyclonal antibody raised against a TUF1 polypeptide. In other embodiments, an antibody composition can include one or more monoclonal antibodies raised against a TUF1 polypeptide. In still other embodiments, an antibody of the antibody composition may be synthesized through recombinant or synthetic methods.

Exemplary antibody targets include for example, TUF1[24-40] (SEQ ID NO:3), TUF1[43-54] (SEQ ID NO:1), TUF1[46-51] (SEQ ID NO:2), TUF1[144-167] (SEQ ID NO:4), or an immunogenic fragment thereof. For example, antibodies may be raised against amino acids 6-20 of SEQ ID NO:3, corresponding to TUF1[24-38], or amino acids 10-24 of SEQ ID NO:4, corresponding to TUF1[153-167].

In another aspect, the present invention also provides a TUF1 polynucleotide—i.e., an isolated polynucleotide that encodes at least a portion of a TUF1 polypeptide. As used herein, a TUF1 polypeptide is a polypeptide having one or more of the functional activities that are described herein. Examples of a TUF1 polynucleotide include an isolated polynucleotide that encodes an amino acid that includes the amino acid sequence of SEQ ID NO:1, SEQ ID NO:2, SEQ ID NO:3, or the complements of such polynucleotide sequences. Other examples of a TUF1 polynucleotide include an isolated polynucleotide that hybridizes, under standard hybridization conditions, to a polynucleotide that encodes an amino acid sequence that includes the amino acid sequence of SEQ ID NO:1, SEQ ID NO:2, or SEQ ID NO:3, or the complements of such polynucleotide sequences. Also included in the present invention are polynucleotides having a sequence identity of at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to a nucleotide sequence that encodes an amino acid sequence that includes the amino acid sequence of SEQ ID NO:1, SEQ ID NO:2, SEQ ID NO:3.

As used herein, “sequence identity” refers to the identity between two polynucleotide sequences. Sequence identity is generally determined by aligning the residues of the two polynucleotides (for example, aligning the nucleotide sequence of the candidate sequence and a nucleotide sequence encoding an amino acid sequence that includes the amino acid sequence of, for example, SEQ ID NO:1 or SEQ ID NO:2) to optimize the number of identical nucleotides along the lengths of their sequences; gaps in either or both sequences are permitted in making the alignment in order to optimize the number of shared nucleotides, although the nucleotides in each sequence must nonetheless remain in their proper order. A candidate sequence is the sequence being compared to a known sequence—i.e., a nucleotide sequence that encodes an amino acid sequence that includes the amino acid sequence of, for example, SEQ ID NO:1 or SEQ ID NO:2. For example, two polynucleotide sequences can be compared using the Blastn program of the BLAST 2 search algorithm, as described by Tatiana et al., FEMS Microbiol Lett., 1999; 174: 247-250, and available on the world wide web at ncbi.nlm.nih.gov/BLAST/. The default values for all BLAST 2 search parameters may be used, including reward for match=1, penalty for mismatch=−2, open gap penalty=5, extension gap penalty=2, gap x_dropoff=50, expect=10, wordsize=11, and filter on.

Also included in the present invention are polynucleotide fragments. A polynucleotide fragment is a portion of an isolated polynucleotide as described herein. Such a portion may be several hundred nucleotides in length, for example about 100, about 200, about 300, about 400, about 500, about 600, about 700, about 800, about 900 or about 1000 nucleotides in length. Such a portion may be about 10 nucleotides to about 100 nucleotides in length, including but not limited to, about 14 to about 40 nucleotides in length.

The polynucleotides of the present invention may be formulated in a composition along with a “carrier.” As used herein, “carrier” includes any solvent, dispersion medium, vehicle, coating, diluent, antibacterial and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions.

By “pharmaceutically acceptable” is meant a material that is not biologically or otherwise undesirable, i.e., the material may be administered to an individual along with a TUF1 polynucleotide without causing any undesirable biological effects or interacting in a deleterious manner with any of the other components of the pharmaceutical composition in which it is contained.

In embodiments in which a TUF1 polypeptide is formulated in a pharmaceutical composition that includes a pharmaceutically acceptable carrier, the polynucleotide may be formulated and administered by methods known to those skilled in the art for delivering therapeutic polynucleotides.

Polynucleotides of the present invention can be inserted into a vector. Construction of vectors containing a polynucleotide of the invention employs standard ligation techniques known in the art. See, for instance, Sambrook et al, “Molecular Cloning: A Laboratory Manual,” Cold Spring Harbor Laboratory Press, 1989. The term vector includes, but is not limited to, plasmid vectors, viral vectors, cosmid vectors, or artificial chromosome vectors. Typically, a vector is capable of replication in a bacterial host, for instance, E. coli. Selection of a vector depends upon a variety of desired characteristics in the resulting construct, such as a selection marker, vector replication rate, and the like. A vector can provide for further cloning (amplification of the polynucleotide), e.g., a cloning vector, or for expression of the polypeptide encoded by the coding sequence, e.g., an expression vector. Suitable host cells for cloning or expressing the vectors herein are prokaryote or eukaryotic cells.

As used herein, an “expression vector” is a DNA molecule, linear or circular, that includes a segment encoding a polypeptide of interest operably linked to additional segments that provide for its transcription. Such additional segments may include promoter and terminator sequences, and optionally one or more origins of replication, one or more selectable markers, an enhancer, a polyadenylation signal, and the like. Expression vectors are generally derived from plasmid or viral DNA, or may contain elements of both.

By “host cell” is meant a cell that supports the replication or expression of an expression vector. Host cells may be bacterial cells, including, for example, E. coli and B. subtilis, or eukaryotic cells, such as yeast, including, for example, Saccharomyces and Pichia, insect cells, including, for example, Drosophila cells and the Sf9 host cells for the baculovirus expression vector, amphibian cells, including, for example, Xenopus oocytes and mammalian cells, such as CHO cells, HeLa cells, human retinal pigment epithelial (RPE) cells, human hepatoma HepG2 cells, and plant cells.

An expression vector optionally includes regulatory sequences operably linked to the coding sequence. The invention is not limited by the use of any particular promoter, and a wide variety of promoters are known. Promoters act as regulatory signals that bind RNA polymerase in a cell to initiate transcription of a downstream (3′ direction) coding sequence. The promoter used can be a constitutive or an inducible promoter. It can be, but need not be, heterologous with respect to the host cell.

The transformation of a host cell with an expression vector may be accomplished by a variety of means known to the art, including, but not limited to, calcium phosphate-DNA co-precipitation, DEAE-dextran-mediated transfection, polybrene-mediated transfection, electroporation, microinjection, liposome fusion, lipofection, protoplast fusion, retroviral infection, biolistics (i.e., particle bombardment) and the like.

Transformation of a host cell may be stable or transient. The term “transient transformation” or “transiently transformed” refers to the introduction of one or more transgenes into a cell in the absence of integration of the transgene into the host cell's genome. Transient transformation may be detected by, for example, enzyme-linked immunosorbent assay (ELISA) that detects the presence of a polypeptide encoded by one or more of the transgenes. Alternatively, transient transformation may be detected by detecting the activity of the protein encoded by the transgene. The term “transient transformant” refers to a cell that has transiently incorporated one or more transgenes. In contrast, the term “stable transformation” or “stably transformed” refers to the introduction and integration of one or more transgenes into the genome of a cell. The term “stable transformant” refers to a cell that has stably integrated one or more transgenes into the genomic DNA. Thus, a stable transformant is distinguished from a transient transformant in that, whereas genomic DNA from the stable transformant contains one or more transgenes, genomic DNA from the transient transformant does not contain a transgene. Methods for both transient and stable expression of coding regions are well known in the art.

The polynucleotides of the present invention may be inserted into a recombinant DNA vector for the production of products including, but not limited to, mRNA, antisense oligonucleotides, and polynucleotides for use in RNA interference (RNAi) (see, for example, Cheng et al., Mol Genet Metab. (2003);80: 121-28). For example, for the production of mRNA, a cDNA comprising a TUF1 polynucleotide as described herein, or a fragments thereof, may be inserted into a plasmid containing a promoter for either SP6 or T7 RNA polymerase. The plasmid is cut with a restriction endonuclease to allow run-off transcription of the mRNA, and the RNA is produced by addition of the appropriate buffer, ribonucleotides, and polymerase. The RNA is isolated by conventional means such as ethanol precipitation. The mRNA can be capped or polyadenylated, for example, prior to injection into a host cell for expression.

In yet another aspect, the present invention provides a method that includes providing a composition comprising a TUF1 polypeptide, wherein the composition is effective to ameliorate at least one symptom or clinical sign of a condition treatable with a neurotrophin when the composition is administered to a subject in need of treatment for a condition treatable with a neurotrophin.

### High Levels of Evolutionary Conservation Implicate an Important Biological Role for Tuf1

The murine tuf1 locus is located on the X-chromosome and includes two exons, spanning approximately 12 kb genomic DNA. The tuf1 gene is predicted to encode a small peptide that contains a signal peptide and three transmembrane domains. Detailed analysis of its peptide sequence identified three possible converting enzyme cleavage sites (KK, RK, or KR). More important, TUF1 shares strong amino acid sequence identities among mammals (97.0% mouse/human and 99.4% mouse/rat) and significant sequence identity between vertebrate and invertebrate orthologs (42.6% mouse/fruit fly). This is a significant level of amino acid identity in comparison to the well-described growth factors such as insulin-like growth factor 1 (IGF-1, 91.5% mouse/human and 98.7% mouse/rat), brain-derived neurotrophic factor (BDNF, 96.8% mouse/human and 99.2% mouse/rat) or nerve growth factor (NGF, 85.4% mouse/human). The N-terminal signal domain and the possible proconvertase cleavage sites suggest that the TUF1 polypeptide is a hormone or a neuropeptide. However, the presence of the transmembrane domains suggests a possible membrane receptor. TUF1 possesses no previously characterized biological function. Data below demonstrate its potentially high impact in the field of stress biology and brain development and function.

### Tuf1 is Expressed in the LHPA Axis

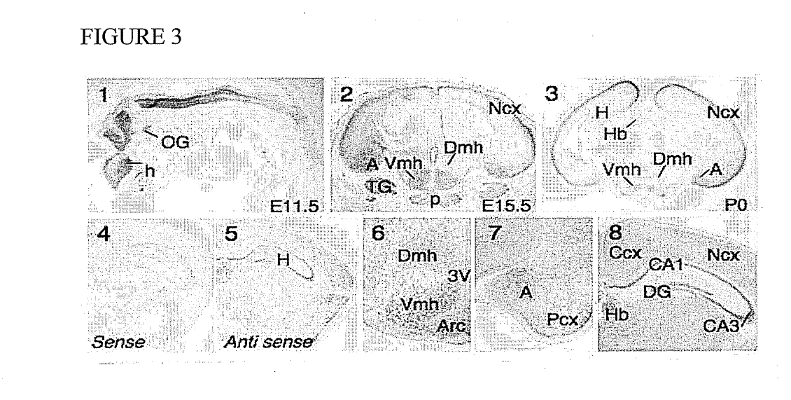

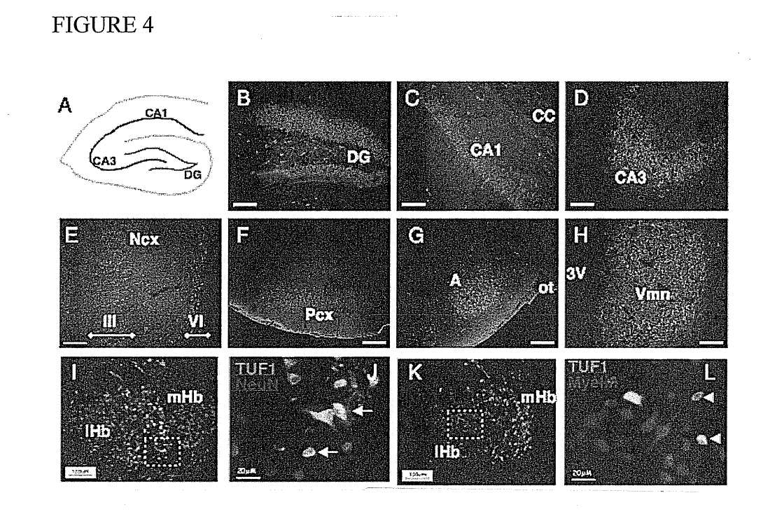

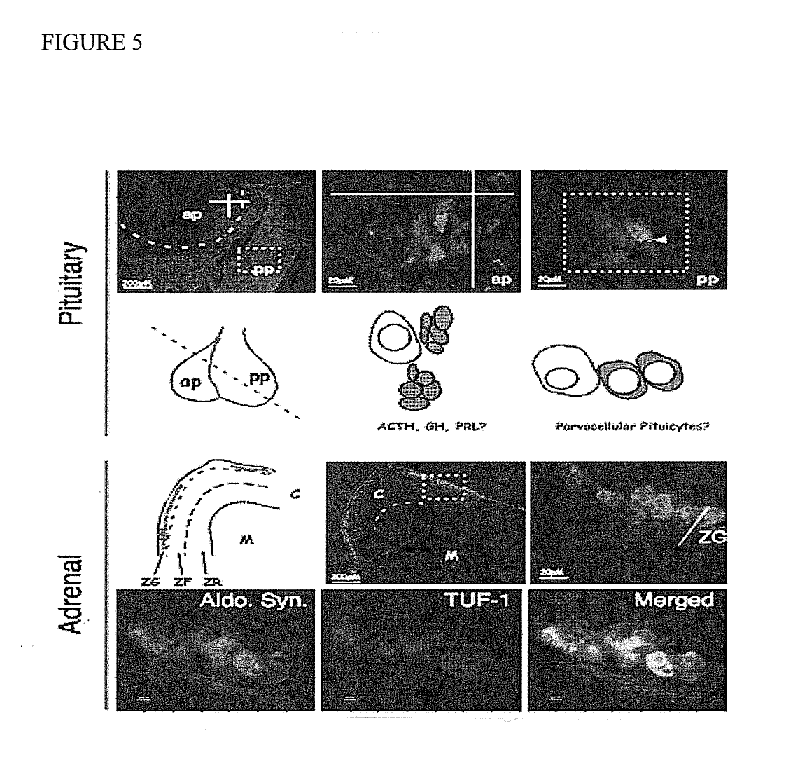

In situ hybridization (ISH) showed tuf1 mRNA in the developing nervous system (FIG. 3, panel 1) with a notable gradient along the developing spinal cord of gestational day (E) 11.5 embryo and in the developing amygdala, VMH, and pituitary of E15.5 brain (FIG. 3, panel 2). In neonatal and adult mouse brains, high levels of tuf1 mRNA are found in VMN, amygdala, hippocampus, habenula, and cortex (FIG. 3, panels 3 through 8). Within the hypothalamus, tuf1 expression is high in selective regions, including the suprachiasmatic nucleus (SCN), the paraventricular nucleus (PVN), the ventromedial nucleus (VMN), and the arcuate nucleus (ARC), but not in the lateral hypothalamus (LHA), the dorsomedial nucleus (DMN) or the mammillary nucleus (MM) (data not shown). These findings are consistent with the expression profile generated by the Allen Institute for Brain Science (Seattle, Wash.). In addition, tuf1 mRNA is also found in the Bed nucleus Stria Terminalis (BnST), the pituitary and adrenal cortex of adult rodents (data not shown). To analyze TUF1 polypeptide expression, an antibody targeting the TUF1 C-terminus (TUF1[153-167]) was developed. The specificity of the antibody was determined by pre-absorption against the synthetic peptide. Protein expression analysis using anti-TUF1 antibody recapitulates the in situ hybridization data, validating expression finding and the specificity of the antibody (FIG. 4). In the CNS of postnatal day 15 male rats, TUF1 polypeptide is found primarily in neurons and in a fewer number of oligodendrocytes (FIG. 4I-L). In the HPA axis, TUF1 polypeptide was also found in the pituitary and the zona glomerulosa (ZG) of the adrenal cortex (FIG. 5). TUF1 overlaps with aldosterone synthase (AS), the P450 steroidogenic enzyme responsible for aldosterone synthesis in the adrenal cortex (FIG. 5, bottom panels), suggesting that TUF1 may regulate salt/water balance by affecting aldosterone secretion.

TUF1 Expression Parallels with the Outgrowth of p75-Positive Nerve Fibers in the Hypertrophic ZG Induced by a Sodium-Deficient Diet

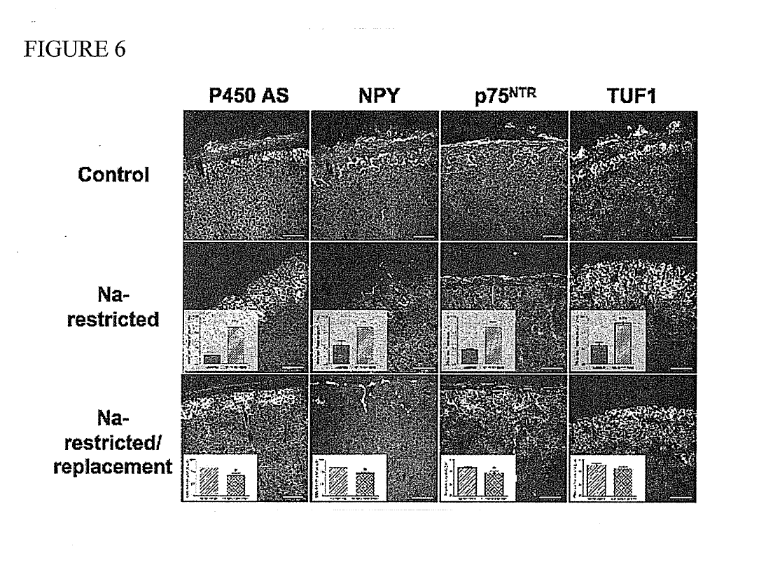

To demonstrate the contribution of TUF1 to the neural basis of low Na⁺-induced ZG hypertrophy, which had not previously been established, male rats were divided into three groups—control, Na⁺ restricted, and Na⁺-restricted/replacement. Each group was fed ad libitum with either control or Na⁺-deficient diet for one week. The Na⁺-restricted/replacement group was fed Na⁺-deficient diet for one week and then control diet for an additional one week. At the end of the diet manipulation, rats were killed and adrenal glands were collected for immunohistology. TUF1 expression expanded concomitant with the p75^{NTR}-positive nerve terminals innervating the ZG in Na⁺-deficient diet and retracted following Na⁺-replacement in Na⁺-deprived rats (FIG. 6). These observations support a role for TUF1 in modulating neurite outgrowth, which may underlie the neural regulation of aldosterone synthesis and secretion.

### Possible Transcriptional Regulators of Tuf1 Expression

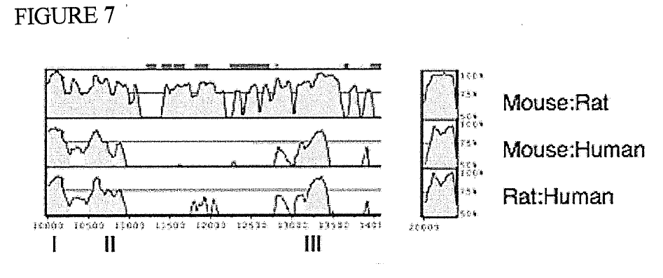

In silico bioinformatics analysis, which aligns the 5′ regulatory region of mouse, rat and human tuf1 genes identified three evolutionary conserved regions (FIG. 7). These regulatory regions may be important in driving tissue-specific and/or stress-regulated tuf1 expression. Bioinformatics analysis that aimed to identify transcriptional factor (TF) binding sites within these evolutionary conserved regions predicted two notable classes of TFs, including steroidogenic factors and cellular differentiation factors (Table 1). These data suggest tuf1 may act as a mediator of cellular responses induced by stress.

TABLE 1
Potential transcription factors regulating tuf1 expression
identified by sequence analysis using MatInpector bioinformatics
(Genomatix Software GmbH, Munich, Germany)

| Conserved | | | |
|---|---|---|---|
| region | Gene | Function | Binding site |
| I, II | MYT1 | MyT1 zinc finger transcription | aacAAGTtaacat |
| | | factor involved in primary | |
| | | neurogenesis | |
| I, II | BCL6 | POZ/zinc finger protein, | gttgcccTAGAaactag |
| | | transcriptional repressor, | |
| | | translocations observed in | |
| | | diffuse large cell lymphoma | |
| II, III | SF1F/FTF | Alpha (1)-fetoprotein transcription | tttcCAAGgtaac |
| | | factor (FTF), liver receptor | |
| | | homologue-1 (LRH-1) | |
| I | GREF/ARE | Androgene receptor binding site | ccgaatcatgctGTCCtca |
| III | GREF/PRE | Progesterone receptor binding site | cccaaatgttaTGTTctgt |
| III | NEUROD1 | DNA binding site for NEUROD1 | tggcCATCtcttt |
| | | (BETA-2/E47 dimer) | |
| I, II, III | LEFF/LEF1 | TCF/LEF-1, involved in the Wnt | gtgacttCAAAgaggat |
| | | signal transduction pathway | |
| I, II, III | PDX1/PDX1 | Pdx1 (IDX/IPF1) pancreatic and | gtaatattgTAATgacagaag |
| | | intestinal homeodomain TF | |
| I, II, III | PARF/TEF | Thyrotrophic embryonic factor | ggtaatattGTAAtgac |

### TUF1 is Localized to Membrane Bound Vesicles and is Potentially Cleaved at the N-Terminus

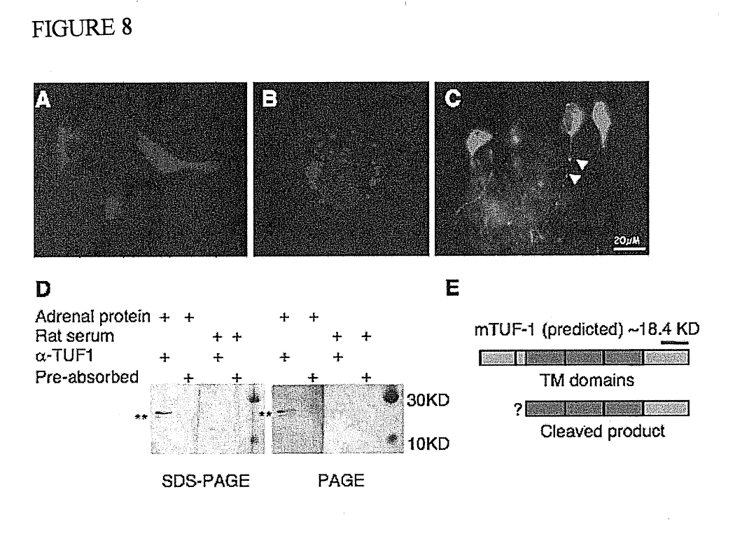

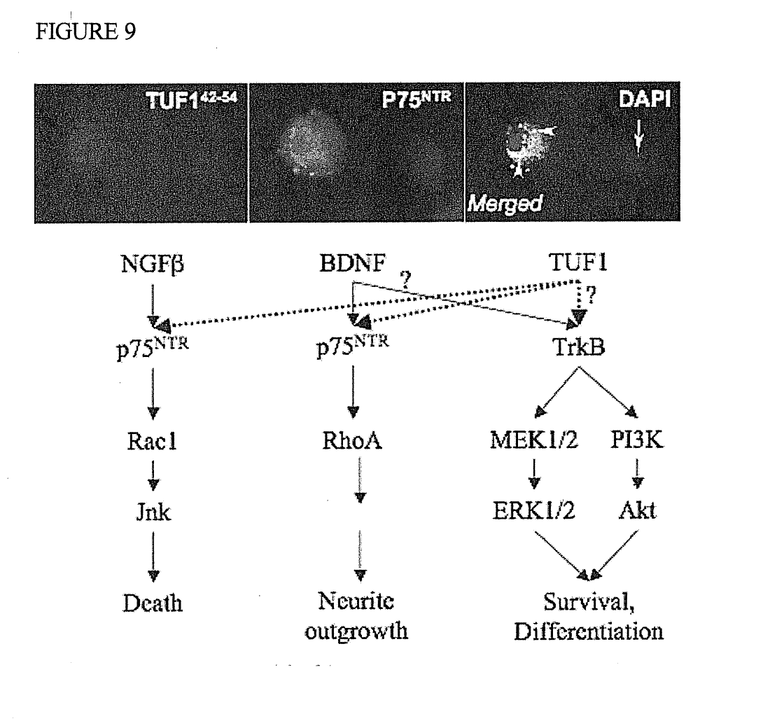

To define the subcellular localization, TUF1 was fused to the red-fluorescent protein (RFP) at the C-terminus and was expressed in cultured cells (COS7). TUF1/RFP fusion protein was found in membrane-bound vesicles, not at the cell membrane. In contrast, RFP (control) alone was found ubiquitously in COS7 cells (FIG. 8A, B). These findings were further confirmed by the observation of vesicle-bound TUF1 in hypothalamic neurons (FIG. 8C, arrows). These data suggest TUF1 is a secreted peptide or a vesicle transporter. In addition, analysis of mouse adrenal protein extract using an anti-TUF1 antibody (C-terminal specific) identified two products in both denatured (SDS-PAGE) and native (PAGE) gels. The molecular weights were extrapolated to be approximately 24.1 kDa (larger) and 22.7 kDa (smaller) products (FIG. 8D), suggesting that TUF1 could be modified by cleavage of the N-terminus to produce a 1.4 kDa smaller protein. Further sequence analysis identified a cleavable N-terminal motif, consisting of 12 amino acids with a predicted 1.4 kDa in size. This motif shares significant sequence homology (6 of 12 amino acids) with the highly conserved p75^{NTR} receptor-binding domain of nerve growth factor (NGF), brain-derived neurotrophic factor (BDNF) and Neurotrophin 3 (NT3). If TUF1 binds the same receptor for NGF, BDNF or NT3, it may regulate signaling cascades that determine neuronal cell fate (FIG. 9). Collectively, these findings suggest that TUF1 may be involved in modulating neuronal development.

### Tuf1 Expression in the HPA Axis has a Circadian Rhythm

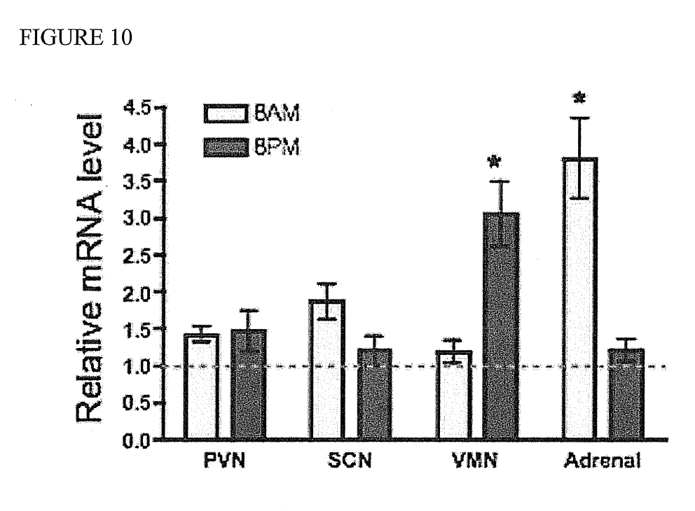

Considering the diurnal cycle of expression and secretion of the well-described hormones in the HPA axis (e.g. CORT, CRF, ACTH), experiments were carried out to determine if tuf1 expression displays a circadian rhythm. Tuf1 mRNA levels were measured in specific hypothalamic nuclei microdissected from adult rat brain using quantitative TAQMAN RT-PCR. Tuf1 expression was found at a high level in the morning (8 a.m.) and at a low level in the evening (8 p.m.) in the suprachiasmatic nucleus (SCN) and the adrenal (FIG. 10). However, a reverse rhythm was found in the VMN (FIG. 10, VMN). If tuf1 is regulated by a wide variety of stressors or CORT, its adrenal expression should be in accord with the VMN. The low adrenal level observed in the evening suggests the presence of an alternative regulator in the adrenal cortex. Nonetheless, these findings suggest TUF1 is likely a hormone and/or neuropeptide. The high evening level of tuf1 expression in the VMN may be driven by energy demand as rats are nocturnal and thus become more active in the evening. It is possible that elevated tuf1 expression in rats in the evening may be induced by a nutritional stress (e.g., hunger).

Tuf1 expression is upregulated in the VMN of animals exposed to cold stress or food deprivation

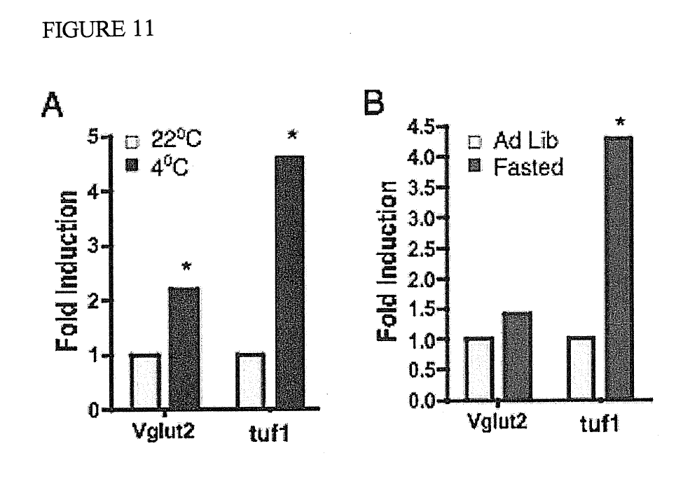

The initial discovery of tuf1 expression in the VMN, which consists of a heterogeneous population of neurons mediating thermoregulation and energy homeostasis, raises the question of whether tuf1 is regulated by a specific VMN function. To answer this, adult mice were exposed to 3-hour cold stress (4° C.) or overnight food-deprivation. Mice were killed at 9 a.m. and tuf1 transcript levels were quantified in the VMN by quantitative RT-PCR. Both stressors increased tuf1 mRNA in the VMN (FIG. 11). Overnight food deprivation increased tuf1 mRNA to about the evening level (8 p.m.) of ad libidum fed rats (FIG. 10, VMN), apparently overriding the normal VMN diurnal rhythm (8 a.m. level). These transcriptional responses suggest that tuf1 is a previously undescribed factor in the HPA axis regulated by energy homeostasis. For example, in view of the afferent projections from the VMN to the PVN, it is possible that TUF1 may relay a signal (e.g., energy deficit), originating in the VMN to the CRF system in the PVN to stimulate a stress response and/or a need to feed.

Gestational and Neonatal Iron Deficiency Causes Long-Term Tuf1 mRNA Reduction in Rat Hippocampus

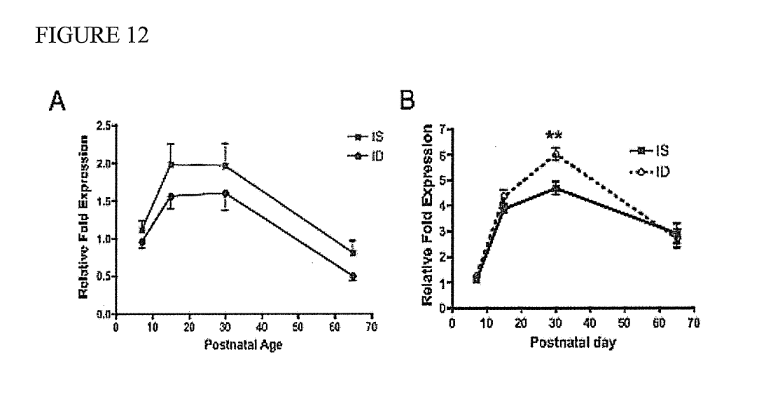

Iron deficiency anemia is a common nutrient deficiency during early-life and is a significant nutritional stressor that alters adrenal glucocorticoid secretion. In humans, early-life iron deficiency impairs cognitive function with acute and long-term effects in spite of prompt iron treatment. Expression of tuf1 in hippocampal neurons raises the question of whether tuf1 expression is altered in iron deficient (ID) and formerly iron deficient rats. Quantitative RT-PCR analysis of tuf1 mRNA following the ontogeny of hippocampal development in the rat revealed that peak tuf1 expression occurs during hippocampal differentiation in both iron sufficient (IS) control and ID rats (postnatal day 15 and 30, FIG. 12A). However, levels of tuf1 mRNA are significantly reduced in rats during and beyond the period of iron deficiency anemia (P65, FIG. 12A). These data show that early-life ID suppresses tuf1 hippocampal expression during and beyond periods of iron deficiency. Concomitant with increased hippocampal GR receptors (FIG. 12B) and reduced glucocorticoid (GC) secretion in ID rats, lower tuf1 expression suggests an overall decline of stress responsiveness in gestational and neonatal ID animals. Taken together, these findings provide additional support for TUF1 modulation of stress responses and suggest that early-life nutritional stress influences tuf1 regulation, possibly altering the “programming” of tuf1 expression.

The above findings suggest at least three possible functions for TUF1, which may not be mutually exclusive. First, TUF1 may mediate how stress alters neuronal differentiation based on the following: its high expression level during neuronal differentiation, its potential role in modulating neurotrophic factor signaling, and its expression may be regulated by transcription factors that promote cellular differentiation (e.g., NeuroD1, Lef-1, Pdx-1, Table 1). Second, TUF1 may be a novel participant in the canonical (GR/CRF/GC) stress response pathway since its expression is induced by acute stressors and also has a diurnal cycle in the HPA axis. The potential binding sites for steroidogenic factors (SF-1/LRH-1, androgen and progesterone receptors) in the tuf1 gene regulatory region (Table 1) further support this possibility. Finally, TUF1 may be a downstream effector mediating energy mobilization in response to a variety of stressful stimuli. In particular, its co-expression with the P540 aldosterone synthase in adrenal glomerulosa, which modulates cardiovascular responses, and its regulation by sodium diet manipulations make TUF1 an attractive candidate for this system.

Thus a TUF1 polypeptide and/or a TUF1 polynucleotide may be used to provide therapeutic benefits to a subject in need of TUF1 therapy. Exemplary therapeutic effects of a TUF1 polypeptide include, for example, neuroprotection in the event of brain injury such as brain injury caused, for example, by seizure, concussion, or trauma; reducing fear and/or anxiety such as may be associated with, for example, post-traumatic stress disorder, social anxiety disorder, etc.; as a drug target for treating cognitive decline-associated conditions; as a drug target for treating obesity and related metabolic disorders such as, for example, peripheral neuropathy; and for treating male fertility.

Diagnostic utility of a TUF1 polypeptide can include, for example, as a diagnostic biomarker for certain conditions. Thus, detection of TUF1 expression in an appropriate sample from a subject can indicate that the subject is at least at risk for developing the condition. TUF1 expression can indicate that an individual is at least at risk for developing neuropathology resulting from, for example, hypoglycemia or genetic predisposition (e.g., Huntington's Disease, Alzheimer's Disease, Parkinson's Disease, dementia, etc.); a fear and/or anxiety disorder such as post-traumatic stress disorder (PTSD) and/or social anxiety disorder; or hypertension.

Inhibition of TUF1 activity also may provide therapeutic benefits. TUF1 activity may be inhibited using a TUF1 polynucleotide such as, for example, a siRNA, RNAi, an antibody, and/or a small inhibitor molecule, in order to reduce TUF1 activity in at least a portion of a subject. For example, a TUF1 inhibitor may reduce hypertension, increase satiety, reduce food intake, and/or reduce obesity in a subject. As used herein, a “TUF1 inhibitor” refers to any compound that measurably reduces at least one TUF1 activity. Methods for producing an antibody that specifically binds a TUF1 polypeptide or a siRNA or RNAi that targets a polynucleotide that encodes a TUF1 polypeptide are known to those skilled in the art.

A subject can be any suitable animal such as, for example, a human, a non-human mammal, a bird, a fish, a reptile, an amphibian, or a marsupial. Thus, suitable subjects include, for example, humans, livestock (e.g., cattle, horses, goats, sheep, and the like), poultry (e.g., chickens, turkeys, and the like), fowl (e.g., geese, ducks, and the like), and companion animals (e.g., dogs, cats, birds, fish, and the like).

The present invention is illustrated by the following examples. It is to be understood that the particular examples, materials, amounts, and procedures are to be interpreted broadly in accordance with the scope and spirit of the invention as set forth herein.

### EXAMPLES

### Example 1

Male Sprague-Dawley rats (250-280 g) were purchased from Charles River, maintained in a 12-hr/12-hr light/dark cycle and were given free access to food and water. All animal protocols were approved by the University of Minnesota IACUC.

Rats were separated into three groups including control, sodium-restricted, and sodium-restricted/replacement (n=6). The control group was fed a control diet containing 0.49% Sodium (TD.96208, Harlan Teklad, Harlan Laboratories Inc., Indianapolis, Ind.). The sodium-restricted group was fed a sodium-deficient diet containing 0.01-0.02% Na (TD.90228, Harland Teklad) for one week. The sodium-restricted/replacement group was fed a sodium-deficient diet for one week and followed with a control diet for one week. At the end of experimental protocols, rats were killed by decapitation and sera were collected from trunk-blood and stored at −80° C.

Dissected adrenal glands were fixed in 4% (w/v) Paraformaldehyde diluted in PBS overnight at 4° C., cryoprotected in 30% (w/v) sucrose/PBS, embedded in a frozen section medium (Neg-50, Thermo Fisher Scientific Inc. Waltham, Ma.), sectioned at 20 μm using a cryostat (VT1000, Leica Microsystems Inc., Bannockburn, Ill.), and stored at −20° C. until further use.

Adrenal sections were equilibrated to room temperature for 10 minutes and rehydrated in Tris saline buffer pH 7.4 (TBS) for 10 minutes. Sections were immersed in 85° C. 20 mM sodium citrate pH 8.0 and cooled to room temperature to unmask antigen. Sections were then permeabilized in 0.2% (v/v) Triton X-100 diluted in TBS for one hour, rinsed in TBS, blocked in BSA (10 mg/ml, Sigma-Aldrich Co., St. Louis, Mo.) for 30 minutes, and incubated in primary antibody diluted in BSA (1 mg/ml) overnight at 4° C. Excess antibody was removed with TBS rinses. Sections were then incubated overnight with Alexa-488 or Alexa-556 secondary antibody (Invitrogen Corp., Carlsbad, Calif.) diluted 1:200 (v/v) with BSA (1 mg/ml). Again, excess antibody was removed with TBS rinses and sections were mounted in aqueous medium containing DAPI (Vector Laboratories Inc., Burlingame, Calif.). Primary antibody included mouse monoclonal anti-P450 aldosterone synthase (1:100 dilution, Millipore MAB6021), rabbit anti-NPY antibody (1:200, Chemicon), rabbit anti-p75^{NTR} antibody (1:100, Cell signaling), and rabbit anti-TUF1 antibody (1:100).

COS7 cells were seeded at 10,000 cells/well onto a 18 mm coverslip in a 12-well plate. Cells were allowed to settle overnight in a cell culture incubator (NuAire NU-8600, NuAire, Inc., Plymouth, Minn.) set at 37° C. and 5% CO₂. Cells were transfected with pCMV-eGFP (Clontech Laboratories, Inc., Mountain View, Calif.) or pCMV-SPORT6-p75 (ATCC) using Fugene HD (Roche Diagnostics Corp., Indianapolis, Ind.) and were incubated overnight. The binding assays were carried out according to Horton et al. with modifications. Synthetic TUF1 peptides (TUF1[43-54] (SEQ ID NO:1) and TUF1[24-40] (SEQ ID NO:3) were generated (via Sigma Genosys, Sigma-Aldrich Co. St. Louis, Mo.) and were conjugated per manufacturer's recommendation with DyLight 549 NHS Ester (Thermo Fisher Scientific, Waltham, Mass.), which have similar spectra to Cy3 dye. Unconjugated dye was removed with a dye removal column (Thermo Fisher Scientific, Waltham, Mass.). Labeled peptides were diluted to 5 nM in 0.15 M Sucrose/PBS and added (1.0 ml/well) to transfected COS7 cells. The binding reaction was incubated in the dark at room temperature for 90 minutes. Cells were rinsed thoroughly with PBS+0.05% Tween-20 to remove unbound peptide. Cells were then fixed with 4% Paraformaldehyde (5 minutes).

Following fixation, cells were permeabilized in PBS+0.1% Tween-20 for 10 minutes, rinsed thoroughly with PBS, and blocked in BSA (10 mg/ml) for 10 minutes. Cells were incubated with rabbit anti-p75^{NTR} antibody (1:10,000 dilution) for 30 minutes. Excess antibody was removed with PBS washes. Cells were then incubated with Alexa-488 goat anti-rabbit antibody (1:500 dilution, Invitrogen). Cells were again rinsed thoroughly with PBS and mounted in Vectashield mounting media plus DAPI (Vector Laboratories, Inc., Burlingame, Calif.).

Digital images were collected with a Nikon confocal microscope (Digital-Eclipse C1 system, Nikon Instruments Inc., Melville, N.Y.) or a Nikon E600 microscope equipped with a CCD camera, and processed with the use of Photoshop (CS3, Adobe Systems Inc., San Jose, Calif.).

All results were presented as means±SEM, n=4/group. Mann-Whitney U-test was performed with C1 set at 95%. Differences were considered significant at P<0.05. Graphs and, statistical calculations were performed with GraphPad Prism (GraphPad Software Inc., San Diego, Calif.).

### Sodium Deficient Diet Induced Neurite Expansion in Adrenal Glomerulosa

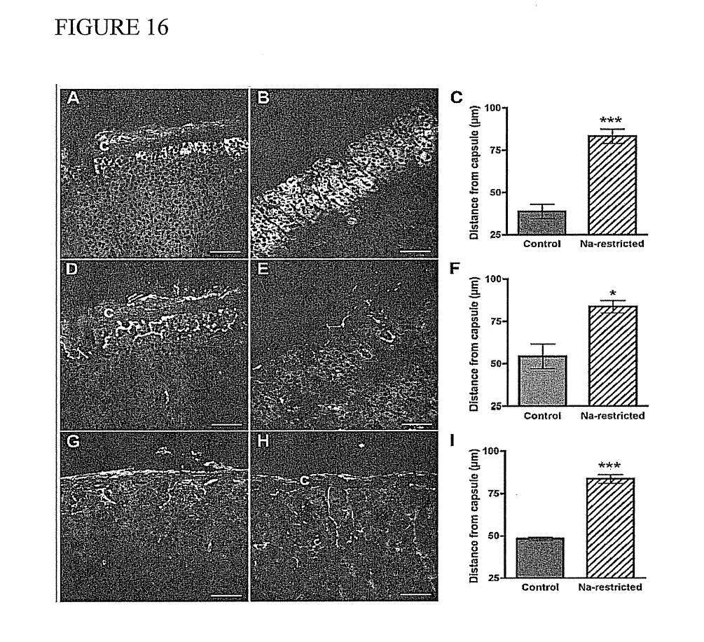

Compared to rats fed a control diet, rats fed sodium deficient diet for one week showed a two-fold increase in glomerulosa zone indicated by the expansion of cells expressing P450 aldosterone synthase (FIGS. 6 and 16). This observation validates the experimental paradigm that aims to induce an adrenal response to salt/water imbalance. To examine whether nerve terminals innervating the glomerulosa zone could be modulated by sodium deficient diet, nerve fibers were stained for neuropeptide tyrosine (NPY) and neurotrophic receptor p75 (p75^{NTR}). Both markers showed nerve terminal expansion along the glomerulosa zone (FIGS. 6 and 16). This observation supports neural modulation of aldosterone production and/or secretion in response to a low sodium diet.

TUF1 Expression Overlapped with P450 Aldosterone Synthase and Induced by Sodium Deficient Diet in Rat Adrenal Glomerulosa

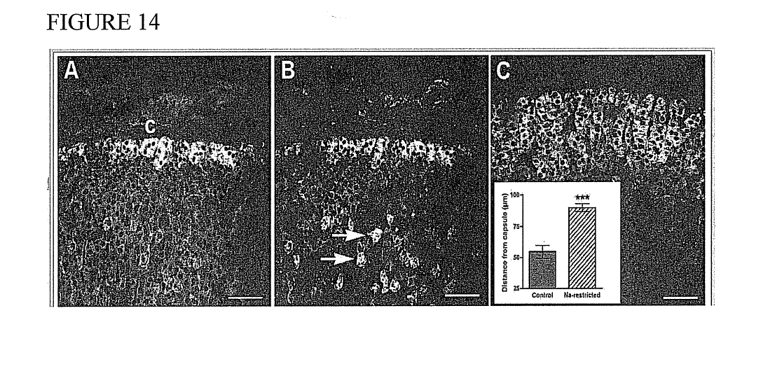

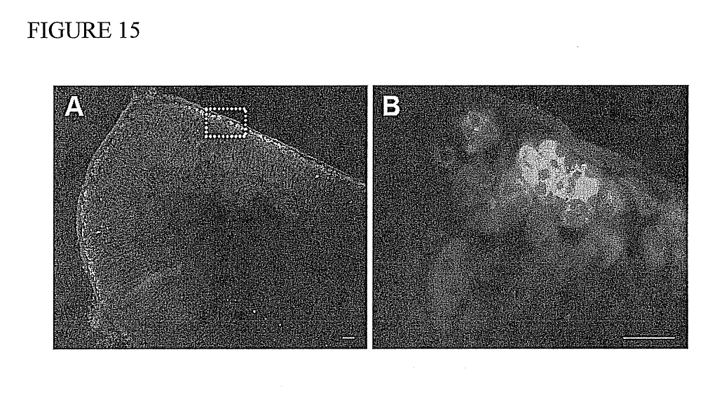

TUF1 is expressed at a high level in the adrenal glomerulosa (FIGS. 14 and 15). To examine whether TUF1 expression can be induced by low sodium diet as shown for P450 aldosterone synthase, antisera against the TUF1 C-terminus was generated and used to stain for TUF1 polypeptide in rat adrenal gland. The antisera were qualified by the absence of TUF1 staining when antibodies were removed by pre-absorption with TUF1 C-terminal peptide (data not shown). In control rats, TUF1 expression was overlapped with aldosterone synthase (FIG. 14A-B) and cell clusters in the adrenal cortex (FIG. 14B). TUF1 was not detected in the adrenal medulla. In rats fed with sodium deficient diet, TUF1 expression expanded significantly (P<0.001) along the glomerulosa zone (FIG. 14C). The magnitude of cell expansion was similar to that observed for NPY or p75^{NTR} (FIGS. 16F, 16I and 14C inset). These findings showed that TUF1 expression is regulated by a low sodium diet

TUF1 Peptide Interacts with p75 Neurotrophic Receptor (NTR) Expressed COS7 Cells

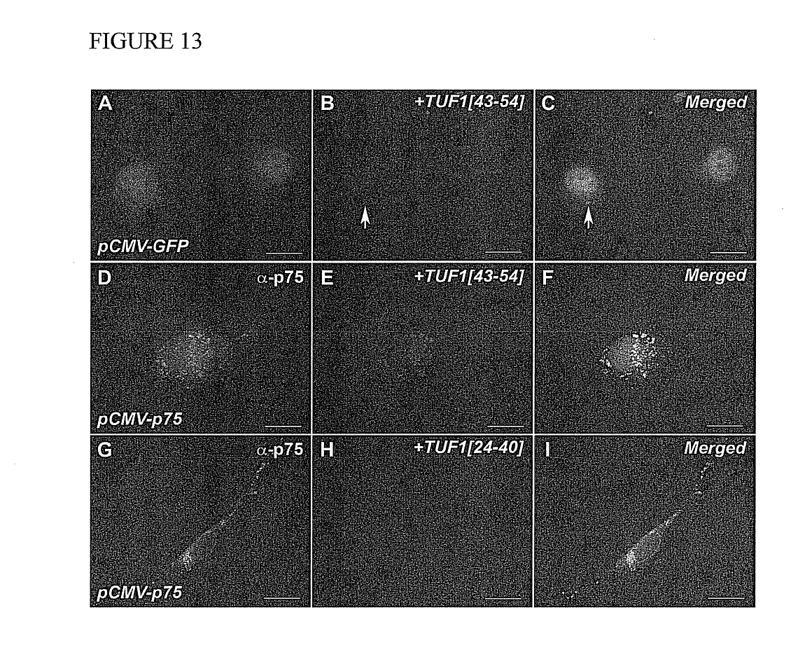

TUF1⁴³⁻⁵⁴ contains a motif that shares strong homology to the highly conserved domain of neurotrophic factors (e.g., NGF, BDNF, and NT3) necessary for interacting with p75^{NTR}, suggesting that TUF1 interacts with p75^{NTR}. To determine TUF1 capability to bind p75^{NTR}, I expressed p75^{NTR} in COS7 cells and performed the binding assay using fluorescence labeled TUF1 peptides. TUF1⁴³⁻⁵⁴ peptide was found to bind the p75^{NTR} (FIG. 13A-F), whereas TUF1²⁴⁻⁴⁰ did not bind the p75^{NTR} (FIG. 13G-I). These findings revealed that the TUF1⁴³⁻⁵⁴ polypeptide interacts with p75NTR, suggesting a novel ligand of the p75^{NTR}. Together with the observation that TUF1 is localized to vesicles, these data support TUF1 peptide to be a secreted factor (FIG. 8A-C) and raise the possibility that TUF1 can promote and/or maintain neurite growth in hypertrophic adrenal glomerulosa.

### Sodium Replacement Induced Regression of Neurite and TUF1 Expression in Glomerulosa

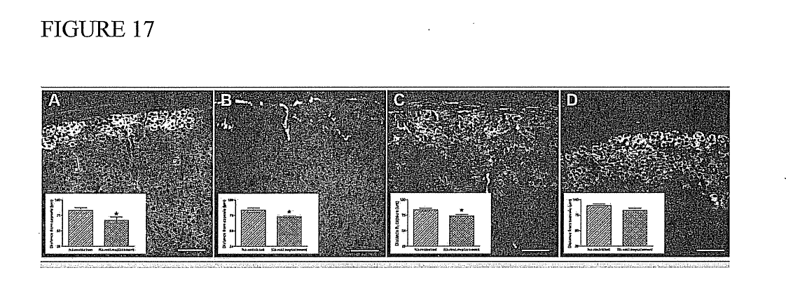

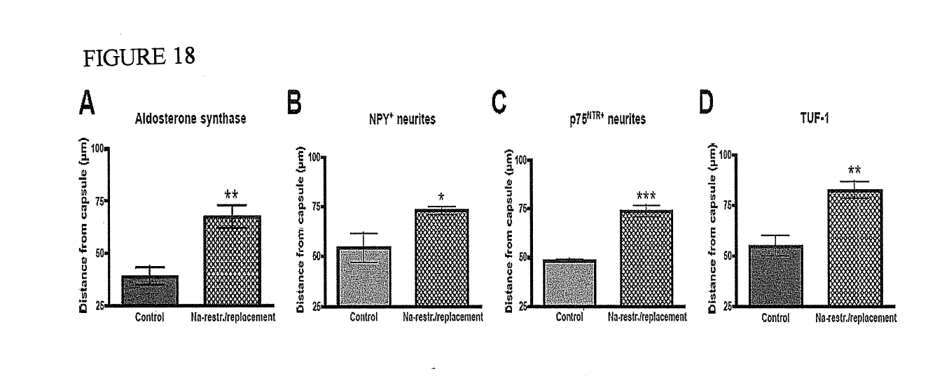

To examine if sodium replacement would restore TUF1 expression and induce neurite regression to a normal state, rats were fed a control diet for 1 week following a week of sodium deficient diet. Examination of P450 aldosterone synthase expression and neurite expansion in the adrenal of these rats showed significant regression of these markers (FIG. 17A-C, P<0.05). In contrast, TUF1 expressing cells showed no regression (FIG. 17D, P=0.16), but had a notable difference in expression level with higher level in glomerulosa (Aldosterone synthase-ir) cells locating proximal to the capsule than in more distal cells (FIG. 17D). These observations suggest that regression of aldosterone synthase expression and neurite length is driven by sodium manipulation. While these factors were still significantly increased in the sodium restricted/replacement group compared to the control group (FIG. 18), normative state would likely be achieved beyond 1-week of sodium replacement. Likewise, variations in TUF1 expression suggest that TUF1 responds to sodium treatment by down-regulating its expression in distal (zona glomerulosa) cells. The low level of TUF1 in these cells could also be the persistent of TUF1 protein. The regression of P450 aldosterone synthase expression, nerve terminals, and TUF1 expression in the adrenal gland of sodium treated rats suggest a physiological relationship among these factors. Their responses to sodium replenishment suggest that their expression would be reduced in animals feeding a high salt diet.

### Summary

We present evidence that neurites terminated in the adrenal glomerulosa zone are dynamically regulated by sodium diets. This finding further supports the role of neural regulation of aldosterone synthesis and secretion. More interestingly, we describe the discovery of a novel factor (TUF1) that is expressed primarily in the zona glomerulosa of the rat adrenal gland. Induction of TUF1 by a low sodium diet suggests a relevant role in regulating aldosterone synthesis/secretion and in turn salt/water balance. Coupled with its interaction with p75^{NTR}, TUF1 may modulate the growth of nerve fibers innervating the adrenal glomerulosa.

### Example 2

Adrenal glands were dissected from adult Sprague-Dawley rats (280 g-300 g) and kept in cold PBS. Following fat removal, adrenal capsules were detached and placed into a few drops of dispersion media (DMEM, 1.0 g/L glucose, 0.32% collagenase (type I, GIBCO, Life Technologies Corp., Carlsbad, Calif.), 4% BSA (Sigma-Aldrich Co., St. Louis, Mo.), 0.1% DNAse (Sigma-Aldrich Co., St. Louis, Mo.). Adrenal capsules were then minced with surgical scissors, transferred to dispersion media and incubated for 90 minutes in a 37° C. and 10% CO₂ cell culture incubator with trituration at 15 minute intervals. Dispersed cells were filtered through a 100 μm wire mesh into wash media (DMEM, 0.4% BSA, 0.28% HEPES) and centrifuged at 200×g for 5 minutes. Following supernatant removal, cells were rinsed in wash media and resuspended in incubation media (wash media+7.65 mM CaCl₂). Dissociated cells were seeded at 75,000 cells/well in a 24-well plate and incubated at 37° C. and 10% CO₂ prior to stimulation. After two hours, cells were incubated in incubation media supplemented with 0 ng/mL, 5 ng/mL, 50 ng/mL, or 500 ng/mL angiotensin II (Sigma-Aldrich Co., St. Louis, Mo.).

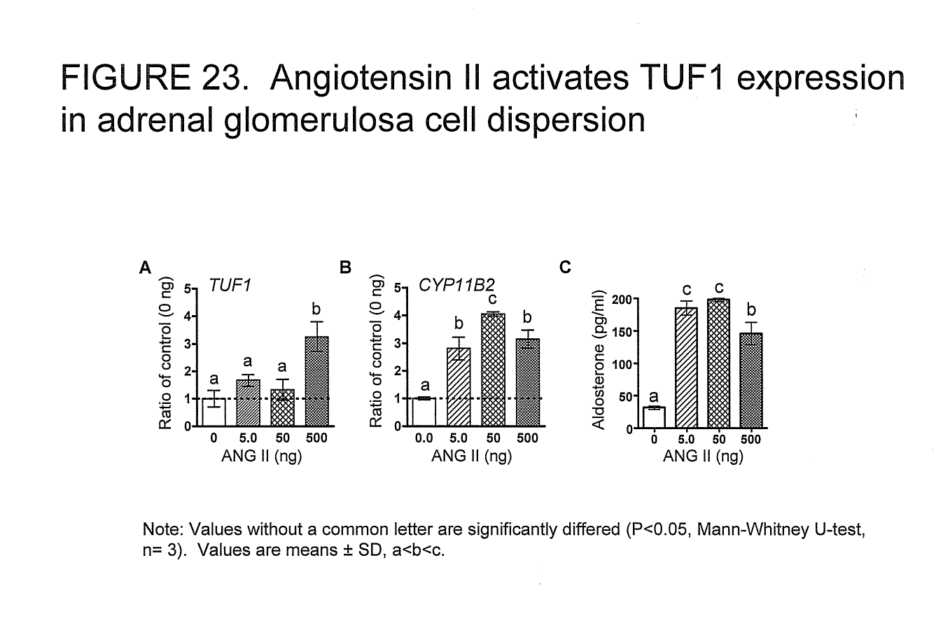

Following overnight incubation, total RNA was isolated from capsular/glomerulosa cells using an RNA-isolation kit (Zymo Research Corp., Orange, Calif.) and concentrations were measured by absorbance at 260 nm (A₂₆₀₁₂₈₀) using a NanoDrop ND-1000 (NanoDrop Technologies, Inc., Wilmington, Del.). 100 ng of total RNA was used to generate cDNA by reverse transcription (High Capacity cDNA RT kit, Applied Biosystems, Life Technologies Corp., Carlsbad, Calif.) per manufacturer recommendation. The resulting cDNA was diluted two-fold to give a final volume of All qPCR experiments were performed with one-half the manufacturer (Applied Biosystems) recommended volume consisting of 4 μl of diluted cDNA, 5 μl 2× TAQMAN qPCR Universal Mix, and 0.5 μl 20× TAQMAN Gene Expression Assay primer/probe mix (CYP11B2, TUF1, and ribosomal protein S18). Thermocyling was carried out according to the manufacturer's protocol (Applied Biosystems) using a MX3000P instrument (Stratagene, La Jolla, Calif.). Results are shown in FIG. 23.

### Example 3

A potential cleaved peptide product of TUF1 consisting of 12 amino acids (SEQ ID NO:1) shares significant sequence homology (six of 12 amino acids) with the highly conserved p75 neurotrophic receptor (NTR)-binding domain of nerve growth factor (NGF), brain-derived neurotrophic factor (BDNF) and Neurotrophin 3 (NT3). This observation leads to the hypothesis that this six amino acid fragment of TUF1 (SEQ ID NO:2) may bind p75 neurotrophic receptor.

COS7 cells were seeded at 10,000 cells/well onto an 18 mm coverslip in a 12-well plate. Cells were allowed to settle overnight in a cell culture incubator (NuAire NU-8600, NuAire, Inc., Plymouth, Minn.) set at 37° C. and 5% CO₂. Cells were transfected with pCMV-eGFP (Clontech Laboratories, Inc., Burlingame, Calif.) or pCMV-SPORT6-p75 (ATCC) using Fugene HD (Roche Diagnostics Corp., Indianapolis, Ind.) and were incubated overnight. The binding assays were carried out according to Horton et al. with modifications. Synthetic TUF1 peptides (TUF1[43-54] (SEQ ID NO:1) and TUF1[24-40] (SEQ ID NO:3)) were generated (via Sigma Genosys, Sigma-Aldrich, St. Louis, Mo.) and were conjugated per manufacturer's recommendation with DyLight 549 NHS Ester (Thermo Fisher Scientific Inc., Waltham, Mass.), which have similar spectra to Cy3 dye. Unconjugated dye was removed with a dye removal column (Thermo Fisher Scientific Inc., Waltham, Mass.). Labeled peptides were diluted to 5 nM in 0.15M Sucrose/PBS and added (1.0 ml/well) to transfected COS7 cells. The binding reaction was incubated in the dark at room temperature for 90 min. Cells were rinsed thoroughly with PBS+0.05% Tween-20 to remove unbound peptide. Cells were then fixed with 4% Paraformaldehyde (5 minutes).

Following fixation, cells were permeabilized in PBS+0.1% Tween-20 for 10 minutes, rinsed thoroughly with PBS, and blocked in BSA (10 mg/ml) for 10 minutes. Cells were incubated with rabbit anti-p75^{NTR} antibody (1:10,000 dilution) for 30 minutes. Excess antibody was removed with PBS washes. Cells were then incubated with Alexa-488-goat anti-rabbit antibody (1:500 dilution, Invitrogen Corp., Carlsbad, Calif.). Cells were again rinsed thoroughly with PBS and mounted in Vectashield mounting media plus DAPI (Vector Laboratories, Inc., Burlingame, Calif.).

TUF1[43-54] polypeptide binds to an unknown receptor expressed in COS7 cell at a low level (FIG. 13B-C, arrow). With cells expressing p75^{NTR} (FIG. 13D), TUF1[43-54] binds more readily to p75 neurotrophic receptor (FIG. 13E-F). In contrast, TUF1[24-40] polypeptide, which does not have any similarity to neurotrophic factors (NGF and BDNF), did not bind cells expressing p75^{NTR} (FIG. 13G-I). These data provide in vitro evidence for the TUF1[43-54] polypeptide binding to p75^{NTR} as well as an unknown receptor expressed in COS7 cells. These observations also suggest that the TUF1[43-54] polypeptide is a potential novel ligand for p75^{NTR} binding.

### Example 4

To gain insight into the binding of TUF1[43-54] peptide and p75^{NTR}, serum deprivation was used as a stressor to COS7 cells transfected with pCMV-SPORT6-p75 expression vector. COS7 cells were seeded at 15,000 cells/well in 12-well plates, transfected with 300 ng pCMV-eGFP (control) or 300 ng pCMV-SPORT6-p75, and incubated overnight. Cells were then incubated in serum-free medium or serum-free medium supplemented with 5 nM TUF1[43-54]. Cells were assessed for Trypan blue dye uptake at 24, 48, and 72 hours.

Immediately after removing growth medium, cells were rinsed with PBS and incubated in 100 μl 0.4% Trypan blue dye diluted in PBS. Images of cells were captured at 10× magnification using an inverted microscope equipped with a CCD camera. Trypan blue-positive cells were counted using Adobe Photoshop CS3 (Adobe Systems Inc., San Jose, Calif.). Graphs and statistics were performed using Prism Graphpad 4.0 (GraphPad Software Inc., San Diego, Calif.).

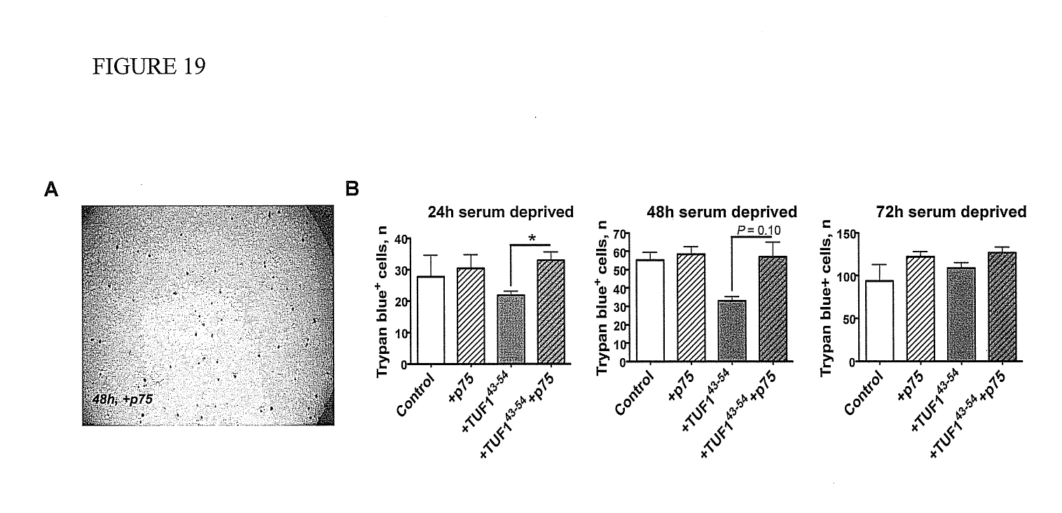

In vitro analysis of COS7 cell survival following serum-deprivation suggests that TUF1[43-54] promotes cell survival (FIG. 19). The presence of p75^{NTR} removed the survival benefit of TUF1[43-54] peptide, while p75^{NTR} alone had no effect on cell survival (FIG. 19B, left and middle panels). This observation suggests that p75^{NTR} acts as a surrogate receptor sequestering TUF1 peptide.

These observations confined that TUF1[43-54] peptide binds to the p75^{NTR} and also suggest TUF1 peptide binds to an unknown receptor to promote the survival effect.

### Example 5

TUF1[43-54] polypeptide was demonstrated to be capable of promoting COST cell survival under serum-deprived environment (Example 4, above). This study aims to determine whether the TUF1[43-54] polypeptide has similar property in primary culture hypothalamic neurons.

Gestational day 16.5 mouse embryos were collected from deeply anesthetized pregnant dam (i.p. injection of 10 mg/Kg Beuthanasia). Embryos were placed in HAM F12+10% Fetal Calf Serum (FCS)+1 mg/ml glucose. Whole brains were dissected and placed in a dish containing Phosphate Buffer Saline (PBS)+1 mg/ml glucose+10 mM HEPES. Hypothalami were dissected from embryonic brains and placed into a conical tube containing 2 ml HAM F12+10 mg/ml Glucose+10 mM HEPES. Cells were dissociated by pipetting and then centrifuged at 200×g for 5 minutes. Cells were suspended in culture medium (HAM F12+10% FCS+10 mg/ml glucose+10 mM HEPES+100 mg/ml antibiotic) and plated at 200,000 cells/25 mm flask. Plated cells were incubated for four days in humidified incubator maintained at 37° C. and 5% CO₂ (NuAire NU-8600, NuAire, Inc., Plymouth, Minn.).

To gain insight into the survival effect of TUF1[43-54] polypeptide, serum deprivation was used as a stressor. Following four days of incubation, cells were incubated in serum-free medium or serum-free medium supplemented with 50 nM TUF1[43-54] polypeptide or 500 ng/ml purified anti-TUF1[43-54] IgG. Cells grew in growth medium with serum supplemental served as control. Cells were assessed for activated-Caspase 3 expression by immunocytochemistry after 48-hour incubation.

Cultured cells were rinsed with PBS (2×), fixed with 4% Paraformaldehyde (5 minutes) and permeabilized with PBS+0.1% Tween-20 (PBST). Following a PBS rinse, cells were incubated in blocking solution (PBS+1% Bovine serum albumin (BSA)+1% normal goat serum). Immediately after blocking, cells were incubated in antibody against activated-Caspase 3 (Rabbit polyclonal, Abcam Inc., Cambridge, Mass.) diluted at 1:5000 in blocking solution for 30 minutes. Excess antibody was removed with PBST rinses (3×). Cells were then incubated with HRP-conjugated goat anti-rabbit antibody (Cell signaling, Danvers, Mass.) diluted at 1:5000 in blocking solution for 30 minutes. Excess antibody was removed by PBST and labeled cells were detected using IMMPACT DAB chromogen (Vector Laboratories, Inc., Burlingame, Calif.) per manufacturer's recommendation.

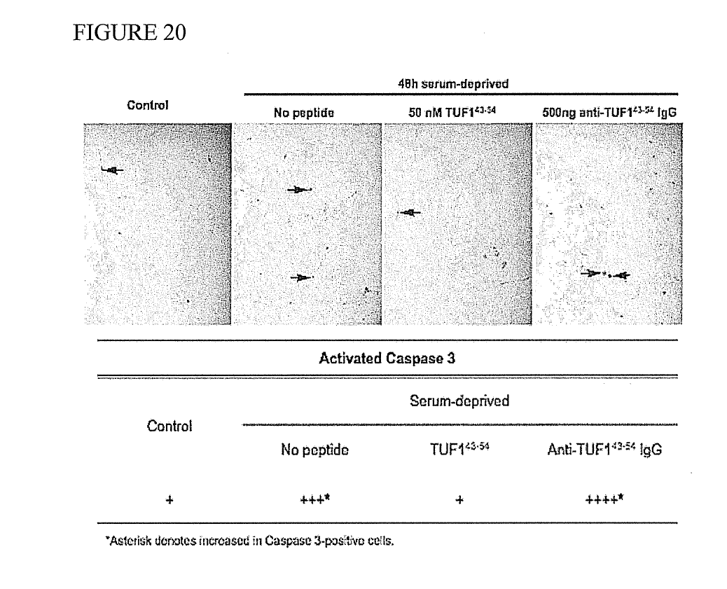

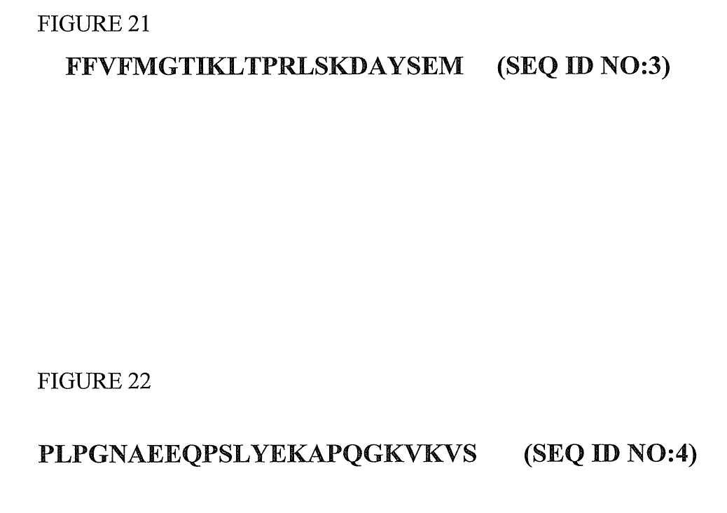

Activated-Caspase 3, a molecular marker of regulated cell death (i.e., programmed cell death/apoptosis), was employed to determine the ability of TUF1[43-54] polypeptide in protecting neurons from undergoing apoptosis. This method was chosen to distinguish gene-regulated cell death from pyknotic (random) cell death. Preliminary qualitative analysis of cell death following serum-deprivation showed fewer cell death occurred in serum-deprived cultures supplemented with TUF1[43-54] polypeptide, whereas increase cell death was observed in culture treated with IgG against TUF1[43-54] polypeptide (FIG. 20). These observations suggest that TUF1[43-54] polypeptide promotes neuronal survival under serum-deprivation, consistent with previous finding. Further analyses will be needed to confirm this observation and to determine if this survival effect also occurs in animal models of stroke and hypoxia.

These preliminary observations demonstrate further the neural protective property of TUF1[43-54] polypeptide. Combined with the finding that stressors (i.e. food-deprivation and cold exposure) upregulate TUF1 expression, these data implicate the potential of TUF1 in mediating neuronal survival under stressful environment.

### Example 6

GT1-1 cell survival assays: GT1-1 cells are immortalized cell line derived from mouse embryonic hypothalamus (a gift from Dr. Richard Weiner, UCSF). GT1-1 cells express TUF1 naturally. To examine the ability of TUF1 peptide to support the survival of these cells under serum deprivation, we performed the following experiments.

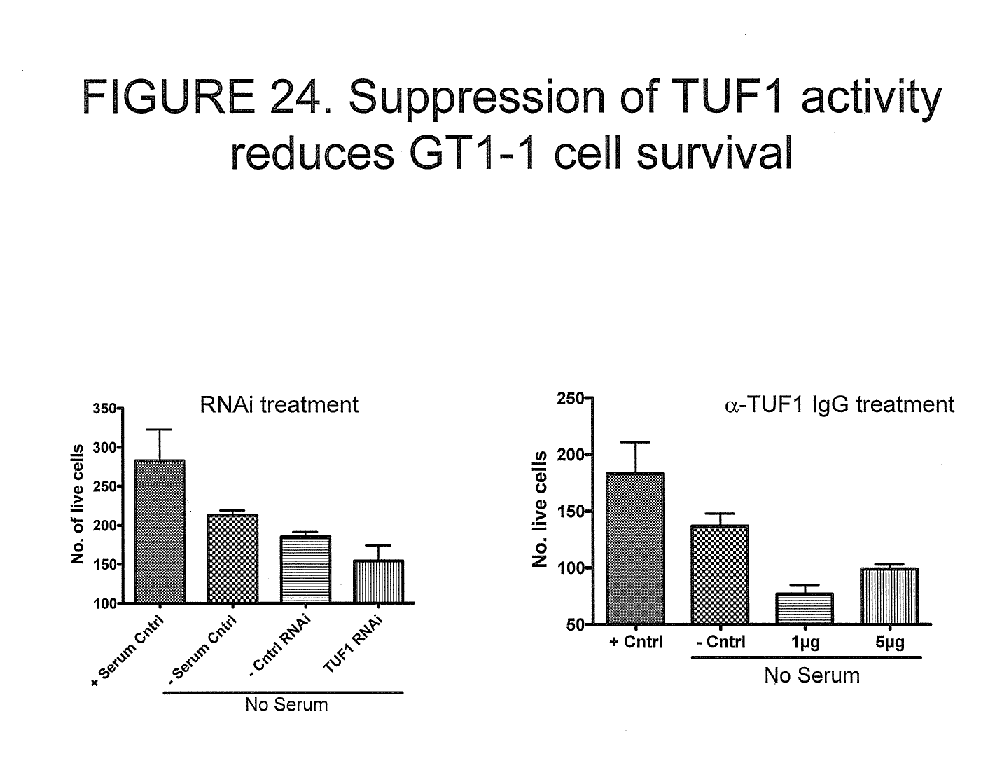

a) Suppression of TUF1 expression by RNAi GT1-1 cells were seeded at 100,000 cells/well in a 12-well plate (as described in Example 1) and incubated for 72 hours at 37° C. supplemented with 5% CO₂. Fresh growth media were added for at least 1 hour before cells were transfected with negative control RNAi or TUF1-specific RNAi (150 picomoles diluted in OptiMEM, Stealth RNAi, Invitrogen Corp., Carlsbad, Calif.) using Fugene6 (1.5 μl/200 μl medium, Roche Diagnostics Corp., Indianapolis, Ind.) and incubated overnight. Cells were then given DMEM without fetal calf serum and incubated for 48 hours. Cells were then lifted from the well by adding Trypsin-EDTA (Invitrogen Corp., Carlsbad, Calif.) and surviving cells were counted by Trypan Blue exclusion using a hemocytometer and an inverted microscope. Values are means±SD, n=3/group. Results are shown in FIG. 24.

b) Repression of TUF1 activity by anti-TUF1 peptide antibody (IgG) GT1-1 cells were seeded at 100,000 cells/well and incubated for 72 hours as described above. Cells were then treated with serum-free media supplemented with 0.0 μg, 3.0 μg, or 5.0 μg of purified anti-TUF1 peptide IgG and incubated for 48 hours. Surviving cells were lifted from wells and counted as described in part (a). Values are means±SD, n=3/group. Results are shown in FIG. 24.

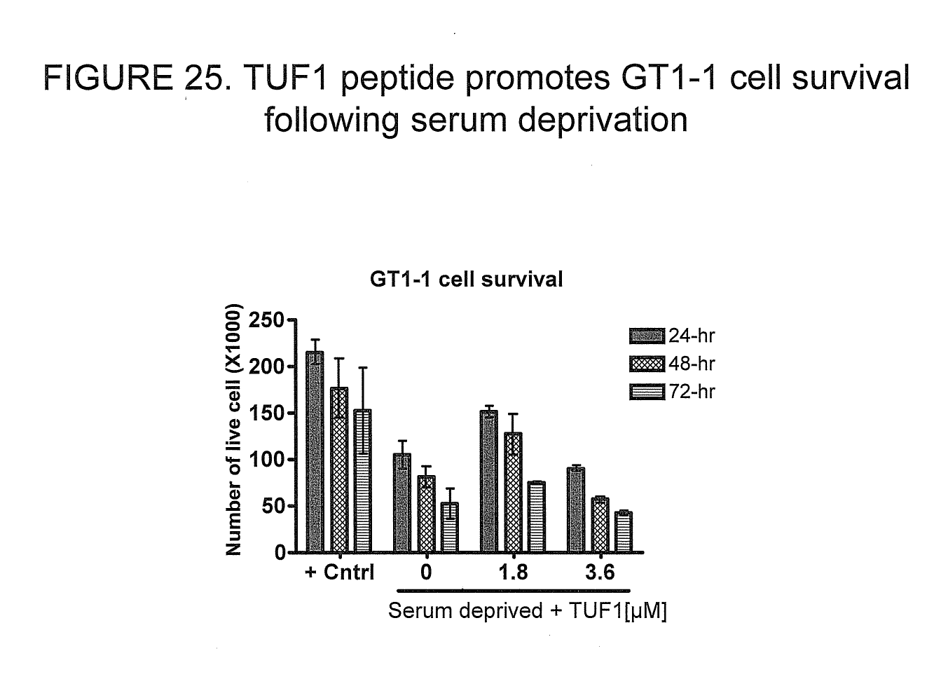

c) Supplementation with TUF1 peptide GT1-1 cells were seeded at 130,000 cells/well in a 12-well plate and incubated for 72 hours. Cells were treated with serum-free media supplemented with 0.0 μg/ml, 2.5 μg/ml (1.8 μM), or 5.0 μg/m (3.6 μM) TUF1 peptide. Surviving cells were counted at 24 hours, 48 hours, and 72 hours of incubation as described in part (a). Values are means±SD, n=3/group. Results are shown in FIG. 25.

### Example 7

GT1-1 cells can be induced to express gonadotropin releasing hormone (GnRH) by retinoic acid supplementation, marking their differentiation into GnRH neurons. To assess TUF1 role in mediating this aspect of neural differentiation, we carried out following experiments.

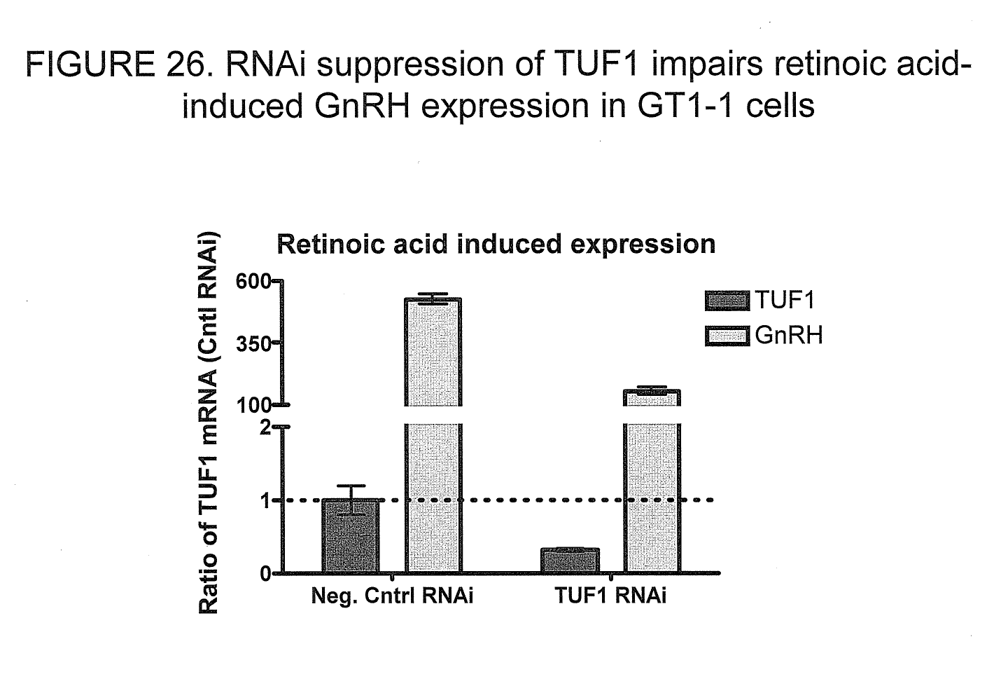

GT1-1 cells were suspended in growth media (Dulbecco's Modified Eagle Media [DMEM], 4.5 g/l glucose, 10% fetal calf serum, 100 unit/ml penicillin G sodium, 100 μg/ml streptomycin sulfate and seeded at 100,000 cells/well in a 12-well plate. Cells were incubated for 72 hours at 37° C. supplemented with 5% CO₂. Fresh growth media without antibiotics were added for 1 hour before cells were transfected with negative control RNAi or TUF1-specific RNAi (200 picomoles diluted in OptiMEM, Stealth RNAi, Invitrogen Corp., Carlsbad, Calif.) using Lipofectamine LTX (2.5 μl/200 μl media, Invitrogen Corp., Carlsbad, Calif.) and incubated for 72 hours. Cells were then stimulated with 1 μM retinoic acid for 30 minutes. Following media removal, cells were lysed with RNA lysis buffer and total RNA were isolated using an RNA isolation kit (Zymo Research Corp., Orange, Calif.). RNA concentrations were measured by absorbance at 260 nm (A₂₆₀₁₂₈₀) using a NanoDrop ND-1000 (Thermo Fisher Scientific Inc., Waltham, Mass.). 100 ng of total RNA was used to generate cDNA by reverse transcription (High Capacity cDNA RT kit, Applied Biosystems, Life Technologies Corp., Carlsbad, Calif.) per manufacturer recommendation. The resulting cDNA was diluted two-fold to give a final volume of 40 μl qPCR experiments were performed with one-half of the manufacturer recommended volume (Applied Biosystems) consisting of 4 μl of diluted cDNA, 5 μl 2× TAQMAN qPCR Universal Mix, and 0.5 μl 20× TAQMAN Gene Expression Assay primer/probe mix (GnRH, TUF1, and ribosomal protein S18). Thermocyling was carried out according to the manufacturer's protocol (Applied Biosystems) using a MX3000P instrument (Stratagene, La Jolla, Calif.). Results are shown in FIG. 26.

### Example 8

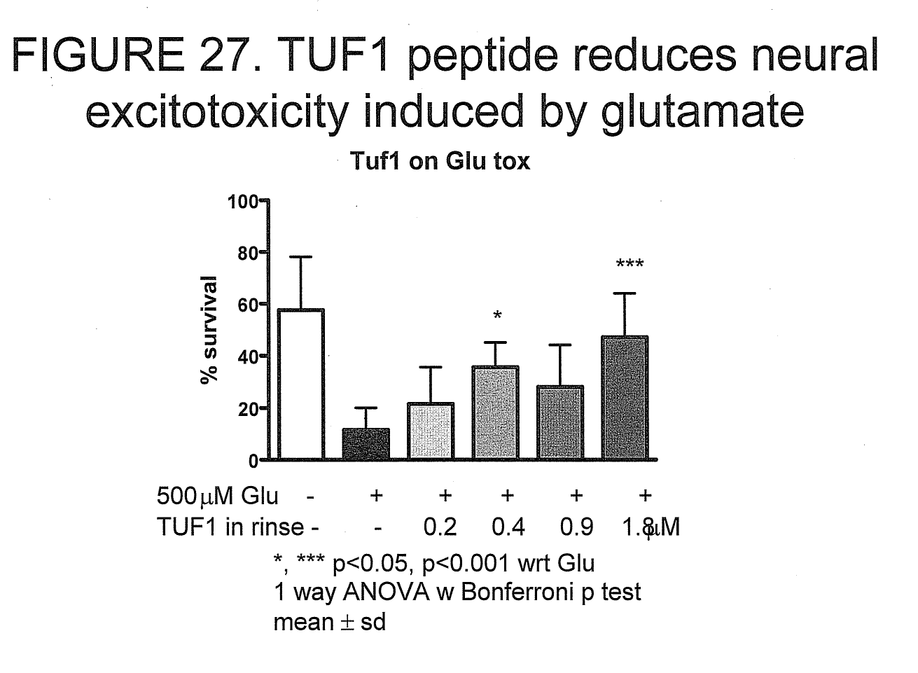

To assess the trophic effect of TUF1 peptide on primary culture neurons, we assayed the effect of TUF1 supplementation on glutamate-induced excitotoxicity in neuronal cultures. Cortical neurons and glia were cultured from newborn mice for seven days. Cultures were then treated with 500 μM glutamate (diluted in N2tox solution, Invitrogen Corp., Carlsbad, Calif.) and incubated at 37° C. for ten minutes. Glutamate was removed from cultures by two washes with EBSS media (Invitrogen Corp., Carlsbad, Calif.). Cultures were then incubated in EBSS media supplemented with 0.0 μM, 0.2 μM, 0.4 μM, 0.9 μM, or 1.8 μM TUF1 peptide for 24 hours. Surviving neurons from cultures were manually counted by Trypan Blue exclusion using an inverted microscope. Values are means±SD, n=6-8/group. Results are shown in FIG. 27.

### Example 9

Assessment of TUF1 mRNA level in limbic hypothalamic structures of Huntington Disease mouse model R6/2

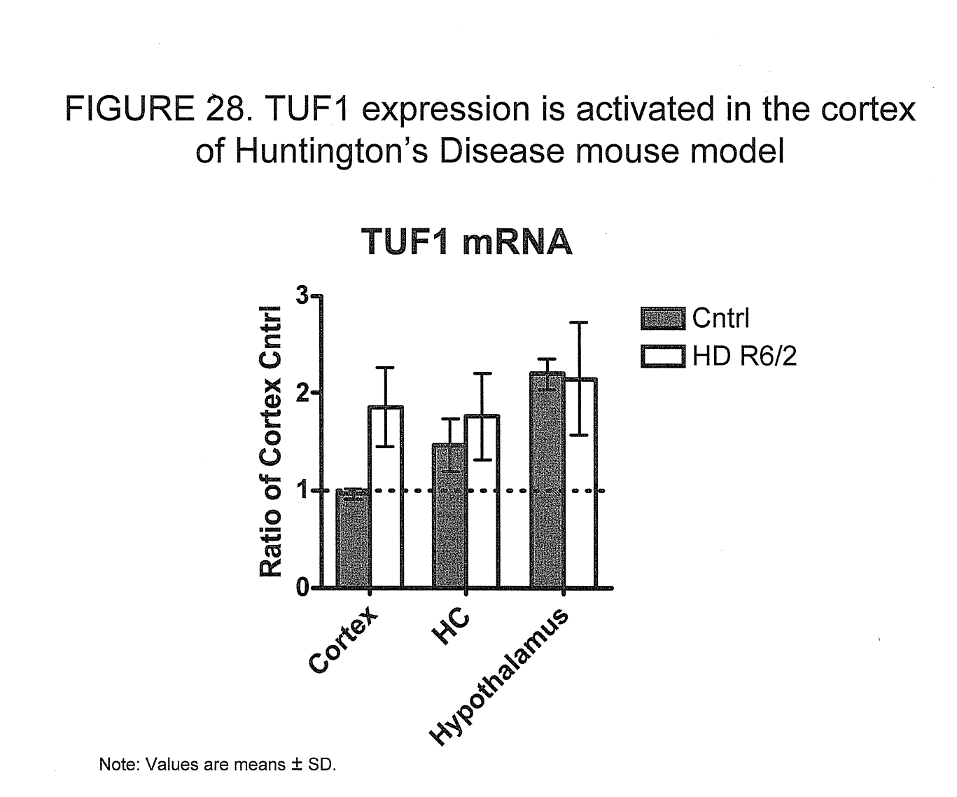

Cortex, hippocampus, and hypothalamus were dissected from 12-week old WT or R6/2 mice and flash-frozen in liquid N₂. Total RNA was isolated from dissected tissues using an RNaqueous isolation kit (Ambion, Life Technologies, Inc., Carlsbad, Calif.) and quantified using Nanodrop ND-1000 (Thermo Fisher Scientific Inc., Waltham, Mass.). cDNA was generated from 1 μg RNA using a High Capacity cDNA RT kit (Applied Biosystems, Life Technologies, Inc., Carlsbad, Calif.) and the resulting cDNA was diluted 10-fold. Quantitative PCR experiments were performed using 1 one-half the manufacturer's recommended volume of TAQMAN Gene Expression Assay and TUF1 TAQMAN probe (Applied Biosystems). Data were collected using a MX3000P instrument (Stratagene, La Jolla, Calif.). Results are shown in FIG. 28.

### Example 10

To determine the effect of early-life nutrient deficiencies on TUF1 expression in the limbic system, we performed the following experiments.

a) Early postnatal hypoglycemia Acute hypoglycemia was induced in P14 male Sprague-Dawley rats with the target blood glucose concentration at <2.5 mmol/l (<40 mg/dl), a value conventionally used to define hypoglycemia in newborn infants. After overnight fasting, human regular insulin (Novo Nordisk Inc., Princeton, N.J.) was injected subcutaneously at 6 IU/kg to half of the rats in a litter (Hypoglycemic group). The other half was injected with equivalent volume of 0.9% saline (control group). Ambient temperature was maintained at 34.0±1.0° C. and fasting was continued for 240 minutes. Blood glucose concentration was measured every 30 minutes using a glucometer (Roche Diagnostics Corp., Indianapolis, Ind.). Hypoglycemia was terminated by intraperitoneal (i.p.) injection of 10% dextrose (200 mg/kg), a dose that corrects brain glucose concentration in hypoglycemic newborn rat. Rats were killed 24 hours later (n=8 per group) using sodium pentobarbital (100 mg/kg, i.p.). The brain was removed and the entire cerebral cortex, hippocampus, and hypothalamus were dissected on ice, flash-frozen in liquid nitrogen and stored at −80° C.

b) Fetal-neonatal iron deficiency Timed-pregnant Sprague-Dawley rats were purchased from Harlan Laboratories (Indianapolis, Ind.). Fetal-neonatal iron deficiency was induced by maintained pregnant dams on an iron deficient diet (3 mg/kg iron, Rx247497) from gestational day 2 to P7, after which time the nursing dams were given the nonpurified IS control diet (198 mg/kg iron, Rx 241632). Both diets were purchased from Harlan Teklad (Harlan Laboratories, Indianapolis, Ind.). IS control animals were given the IS diet throughout the experiment. Litters were culled to 8 pups/litter, all pups were weaned at P21 and fed IS diet for the duration of the experiment. All animal experiments were carried out with the approval of the University of Minnesota Institutional Animal Care and Use Committee. Postnatal day 7, day 15, day 30, and day 65 male rats were killed using sodium pentobarbital (100 mg/kg, i.p.). Brains were removed and bisected along the midline. Hippocampus was dissected and flash-frozen.

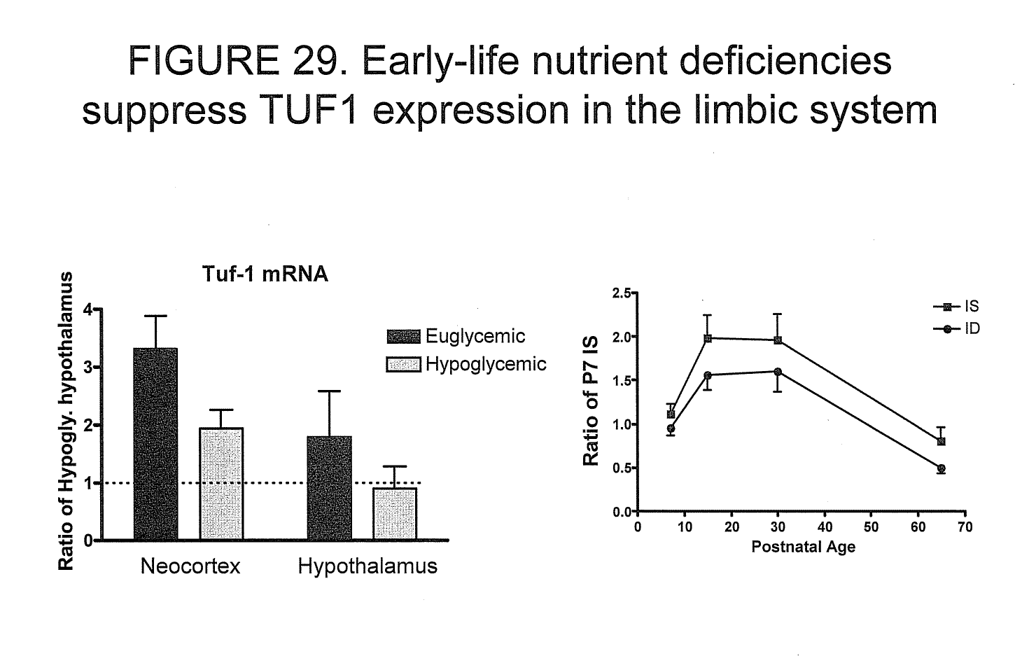

c) Quantitative PCR Total RNA was isolated from dissected hippocampus, cortex or hypothalamus using an RNA-isolation kit (Stratagene, La Jolla, Calif.). 2 μg of total RNA was used to generate cDNA using a High Capacity cDNA RT kit (Applied Biosystems, Inc., Life Technologies Corp., Carlsbad, Calif.) and the resulting cDNA was diluted seven-fold. Quantitative PCR experiments were performed using one-half the manufacturer's recommended volume of TAQMAN Gene Expression Assay and custom-designed rat TUF1 TAQMAN probe (Applied Biosystems). Data were collected using a MX3000P instrument (Stratagene, La Jolla, Calif.). Results are shown in FIG. 29.

### Example 11

The high level of TUF1 expression in the adult rat amygdala (FIG. 4G) suggests a potential role in mediating amygdala-based fear responses. We evaluated the effect of central TUF1 administration on fear response in rats.

a) Intra-cerebroventricular (ICV) Cannulation Male Sprague-Dawley rats weighing 250-400 grams were anesthetized with ketamine (100 mg/kg) and xylazine (7 mg/kg) and placed in a stereotaxic device (David Kopf Instruments Corp., Tunjunga, Calif.) using atraumatic ear bars. An incision of the skin overlying the skull was made and anchor screws were inserted into the skull. Burr holes were drilled to allow the guide cannula to pass into the brain. The guide cannula (22-gauge, C313G; Plastics One, Roanoke, Va.) was placed at 0 mm posterior, 1.2 mm lateral and 3.5 mm ventral to bregma. The cannula was affixed using epoxy glue (Loctite 444; McMaster-Carr, Chicago, Ill.) followed by dental acrylic (Hygienic perm reline & repair resin, Type II, Class I; Henry Schein, Inc., Melville, N.Y.). The cannula was sealed and kept open by insertion of a ‘dummy’ cannula.

b) TUF1 Peptide Infusion 0 μg, 1 μg, or 10 μg TUF1 peptide diluted in artificial cerebrospinal fluid (aCSF, Harvard Apparatus, Holliston, Mass.) was injected into the brain through the in-dwelling cannulae immediately prior to behavioral measurement (described below). The infusion procedure involved removal of the “dummy” cannulae from the implanted guide cannulae and insertion of internal cannulae. These were attached via polyethylene tubing to 10 μl Hamilton syringes fitted to a microinfusion pump (Harvard Apparatus, Holliston, Mass.). A volume of 2 μl was infused at a rate of 1 μl/min. The internal cannulae were left in place for one minute following infusion to ensure adequate diffusion. The dummy cannulae were then replaced, and the rats were brought to chambers for behavioral testing.

c) Behavioral Testing

I. Food Consumption

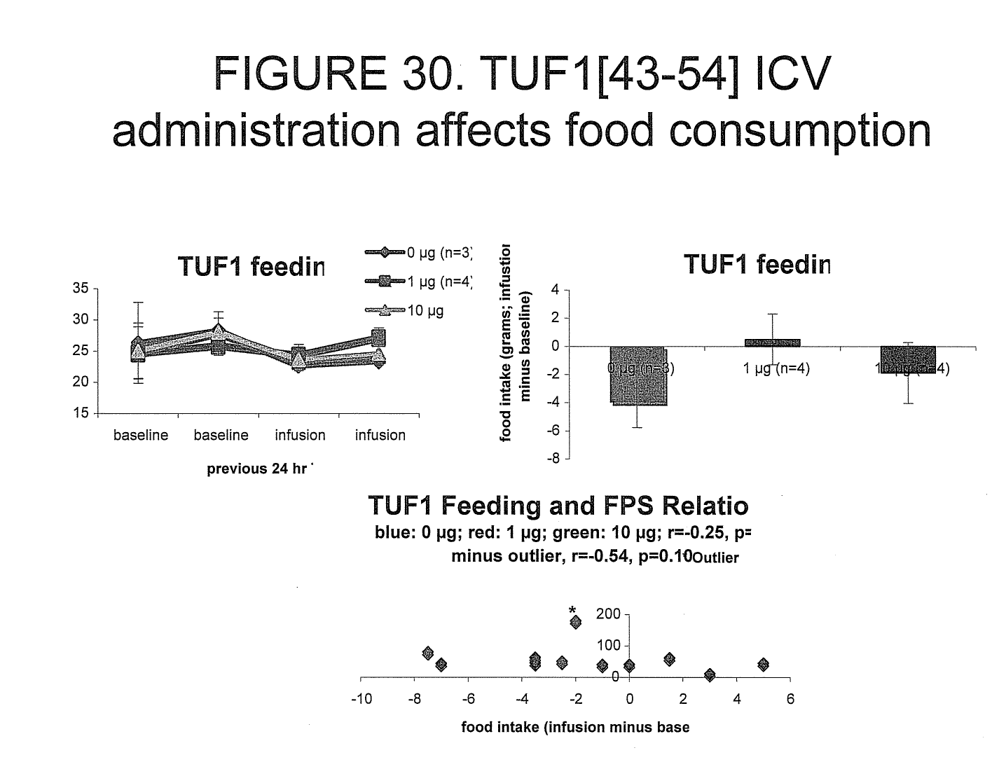

Food intake, in grams, was measured by weighing food in the hopper daily for the two days prior to drug infusion and for the drug infusion days, and calculating the amount eaten by subtracting each day's food weight from that of the previous day. Results are shown in FIG. 30.

II. Fear-Potentiated Startle

For fear potentiated startle, shock sensitization, and prepulse inhibition testing, animals were trained and tested in four identical 7.5 cm×9 cm×17 cm stabilimeter devices. Each stabilimeter consisted of a Plexiglas cage, which rested on four compression springs and was located within a ventilated sound-attenuating chamber. Cage movement resulted in displacement of a Type 338B35 accelerometer (PCB Piezotronics, Inc., Depew, N.Y.) attached to each cage. The resultant voltage of the accelerometer was proportional to the velocity of the cage displacement. This signal was amplified by a signal-processing unit (No. 482820; PCB Piezotronics, Inc. Depew, N.Y.). An InstruNet 100b board (GW Instruments, Inc., Somerville, Mass.) interfaced to a Dell computer digitized the voltage output of the accelerometer on a scale of 0-100 units. Startle amplitude was defined as the peak accelerometer voltage that occurred during the first 200 ms after onset of the startle stimulus. High-frequency speakers (Super Tweeters, range 5-40 kHz; No. 40-1310b, RadioShack Corp., Fort Worth, Tex.) located 5 cm behind each cage delivered the startle stimuli. The startle stimuli were 50 millisecond (ms) (5 ms rise and decay times) bursts of white noise (low pass, 22 kHz) at 95 dB and 105 dB. The ventilation fans of the sound-attenuating chamber elevated background noise to 60-65 dB. The foot shock unconditioned stimulus (US) was a 0.5 second, 0.8 milliAmp (mA) constant current scrambled shock, delivered by a shock generator (no. SGS-004; BRS/LVE, Laurel, Md.) through the four bars that made up the bottom of the stabilimeter. Shock intensity was measured with a 1-kΩ resistor across a differential channel of an oscilloscope in series with a 100-kΩ resistor connected between two floor bars in each cage. Current was defined as the RMS voltage across the resistor and calculated in mA as 0.707×0.5 ×peak-to-peak voltage. The conditioned stimulus (CS) was a 7.5 second band-pass filtered noise, raised to a sound pressure level 5 dB above background noise (65-70 dB), with high and low cutoffs set at 4 kHz and 24 dB per octave attenuation. The noise was generated by the computer and delivered through a low-frequency speaker (Model no. 40-1024A, RadioShack Corp., Fort Worth, Tex.) situated 15 cm from the cage. Stimulus presentation and data recording were managed with Matlab software (The MathWorks, Inc., Natick, Mass.).

To acclimate the rats to the apparatus and startle stimuli and to measure levels of baseline startle, rats underwent two days of startle testing. After a five minute acclamation period, they were presented with 40 startle stimuli, 20 each at 95 dB and 105 dB intermixed pseudorandomly, separated by a 30 second interstimulus interval.

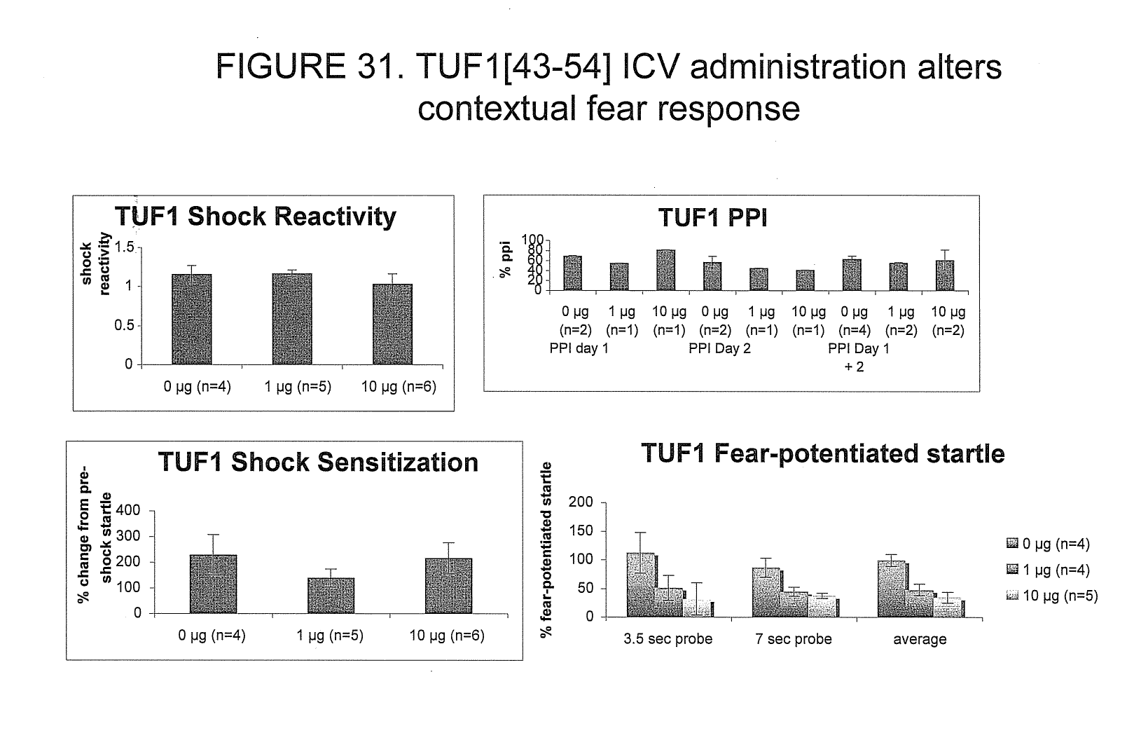

On the third and fourth days, rats underwent TUF1 or vehicle ICV infusions followed immediately by training in a fear conditioning paradigm. After a five minute acclamation period, 10 startle stimuli were presented with a 30 second interstimulus interval. Then, rats were presented with 12 trials consisting of the 7.5 second tone CS co-terminating with the 0.5 second shock US, with a 90-180 second variable intertrial interval. After the CS-US pairings, rats were again presented with 10 startle stimuli with a 30 second interval. On the fifth day, fear conditioning was tested. After a 5 minute acclamation period, 30 startle stimuli at each of two startle intensities (95 dB and 105 dB) were presented to bring the startle response to a stable baseline. This was followed immediately by 20 presentations of the startle stimulus at each of the intensities in the presence of the CS, with 10 of the startle stimuli presented 3.5 seconds after the onset of the CS, and the other 10 presented 7.0 seconds after CS onset (at the time shock would have been presented during training). These CS-startle pairings were intermixed with 10 startle presentations in the absence of the CS. Fear-potentiated startle is calculated as difference or percent change in mean startle amplitude in the presence versus the absence of the CS. Results are shown in FIG. 31.

III. Shock Sensitization of Startle

After shock exposure, rats show a potentiated acoustic startle response. This shock sensitization of the startle response has been proposed as a way to study contextual fear conditioning (Richardson R., 2000. Shock sensitization of startle: learned or unlearned fear? Behavioural Brain Research 110(1-2): 109-117). Shock sensitization of startle was measured as the percent change in mean startle amplitude from the last four startle stimuli before CS-US pairings to the first four startle stimuli after the pairings on the first training day. Results are shown in FIG. 31.

IV. Prepulse Inhibition of Startle

Prepulse inhibition (PPI) of the startle reflex was used to measure sensorimotor gating, which is the regulation of transmission of sensory information to the motor system. Humans with neurological disorders such as schizophrenia, Huntington's disease, and obsessive-compulsive disorder demonstrate sensorimotor gating disruptions. In prepulse inhibition, a mild stimulus (prepulse) suppresses the response to a strong startle-eliciting stimulus when the prepulse precedes the startle stimulus by a brief duration (10-500 ms in mammals). After ICV infusion of TUF1 or vehicle, PPI was measured in the same chambers as were used for fear conditioning and shock sensitization testing. After a 5 minute acclamation period, six 115 dB, 40 ms startle stimuli were presented at 30 second intervals to habituate the startle response. Then, five prepulses, each at either 68 dB, 71 dB, or 77 dB (20 ms white noise) were followed 100 ms later with a 115 dB startle (pulse) stimulus. The prepulse-startle pairings were presented with variable 8-23 second intertrial intervals, and were intermixed with five startle-alone trials. Percent PPI was calculated by 100 [(startle alone amplitude)−(prepulse+startle amplitude)]/(startle alone amplitude). Results are shown in FIG. 31.

### Example 12

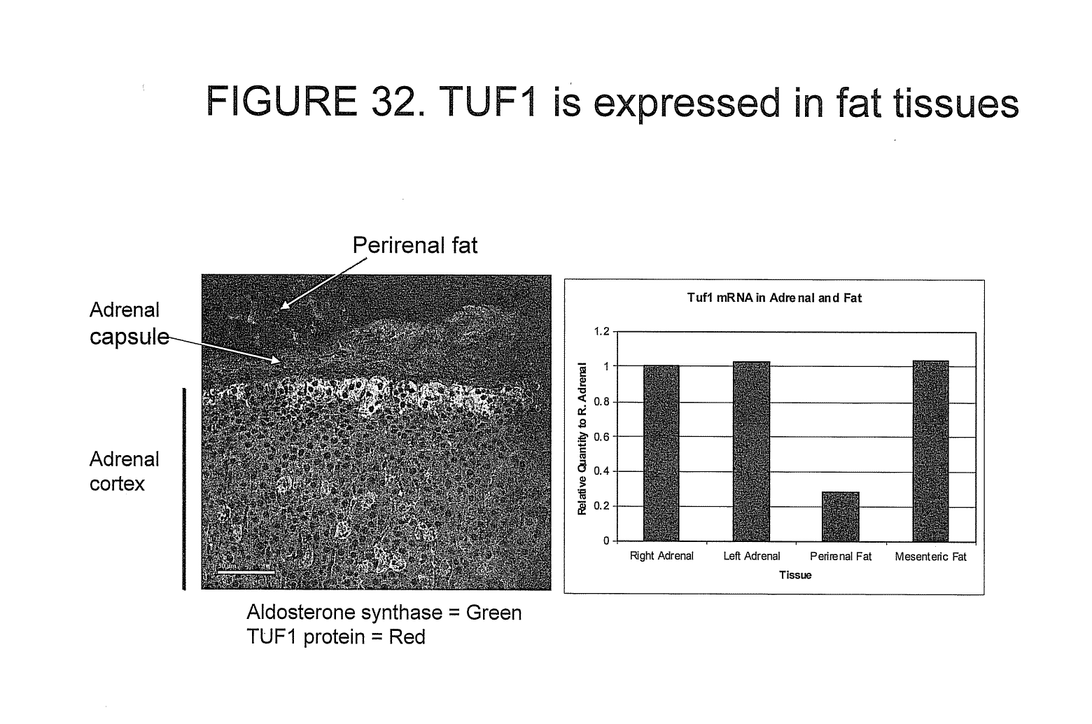

TUF1 expression in fat tissues was assessed by immunohistochemistry using polyclonal antibody raised against TUF1[153-167] (Sigma Genosys, Sigma-Aldrich Co., St. Louis, Mo.) and quantitative PCR using customized TUF1 TAQMAN probe. Based on TUF1 potential role in mediating nerve innervation in the adrenal glomerulosa, TUF1 expression in fat depots may have a similar function regulating fat tissues innervation that may contribute to adipocyte growth and metabolic activity. If correct, TUF1 can be a candidate target for a novel strategy to prevent the development of obesity and associated metabolic disorders. Left panel shows labeled (green) aldosterone synthase, marking the adrenal zona glomerulosa. Right panel shows mRNA levels of TUF1 relative to TUF1 expression in the right adrenal. Tissues were collected from adult rats. Results are shown in FIG. 32.

### Example 13

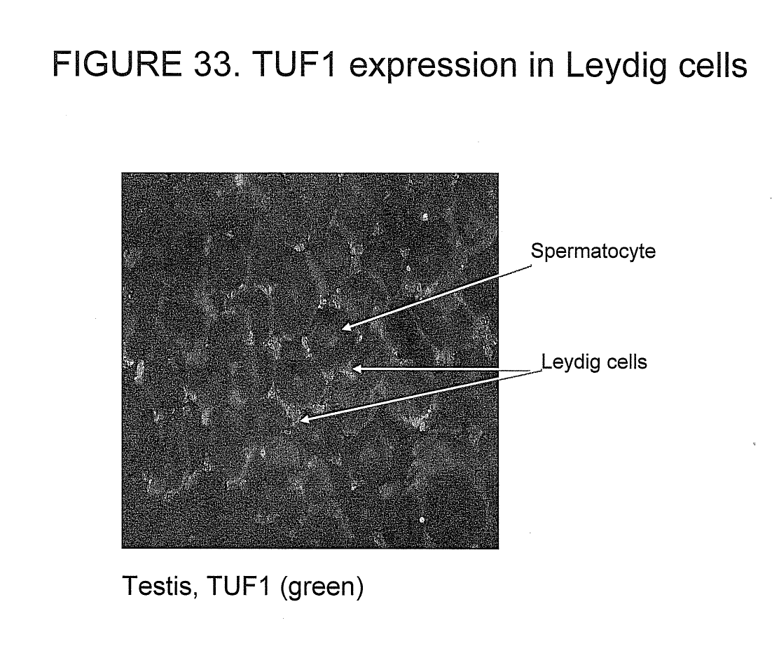

TUF1 expression was detected in Leydig cells of male rats using polyclonal antibody raised against TUF1[153-167] (Sigma Genosys, Sigma-Aldrich Co., St. Louis, Mo.). Leydig cells synthesize and secrete male steroid hormone testosterone. Results are shown in FIG. 33.

### Example 14

Three highly conserved regions within 10 kb upstream of transcription start site have been defined based on in silico DNA sequence analysis (FIG. 7A). These conserved regions may have important regulatory roles. Potential transcription factor binding sites have also been identified using weighed matrices of binding sites (Table 1). In vitro transfection assays will be carried out to confirm the ability of a specific transcription factor to transactivate reporter constructs (FIG. 7B). Promoter region-I, II or III can be subcloned into an expression vector containing a luciferase reporter with a minimal promoter. These constructs can be co-transfected into Y1, PC12 or COST cells with specific transcription factor driven by a constitutive promoter (e.g., pCMV/SF1 or pCMV/LRH1). In particular, Y1 cells are preferred for promoter-containing steroidogenic factor binding site, because they express SF1 transcription factor endogenously. Luciferase activity will be measured to determine the capability of transcription factor to transactivate selective promoter. The reporter construct with minimal promoter will be used as a negative control. Data from these studies will provide insights into the regulation of tuf1 expression in terms of tissue-specific factors and condition-specific factors.

### Example 15

Identified regulatory elements that contribute to tissue-specific tuf1 expression will be cloned into a construct containing an eGFP/Cre recombinase gene cassette (Addgene, Mass.). Following in vitro validation by co-transfection of cultured cells (Y1, PC12), these constructs will be used to generate transgenic mice. Blastocyst injections will be contracted to the University of Minnesota Transgenic Animal Model Core. Transgenic animals will be genotyped by tail tissue and PCR amplification using tuf1 For and eGFPRev primers, which will be designed to produce a 200-300 bp product. Transgenic founders will be crossed into C57B1/6J mice to identify germ line transgenes and to maintain mice in this genetic background. Multiple transgenic founder lines of each construct will be examined for eGFP expression to rule out possible ectopic expression resulting from position-effect insertion. These transgenic animals will be critical in generating tissue-specific gene knockout animal models, facilitating the analysis for specific requirement of TUF1 during development and/or in adult function.

### Example 16

Approaches utilizing both genetic and cell biology tools will be used to identify TUF1 downstream effector(s). UAS/Dtuf1/dsRNA transgenes will be created to knockdown tuf1 activity in Drosophila, which will be used as a model to screen for non-complement mutations. These mutations will be tuf1 alleles or genes that function in the same genetic pathway (e.g., receptor, effector). Likewise, enhancers or suppressors of tuf1/dsRNA-induced phenotype(s) will lead to the identification of regulatory factors (e.g., transcription factors) or functional co-regulators (e.g., antagonist), respectively. As an alternative approach, a modified version of expression cloning technique and fluorescence resonance energy transfer (FRET) technique will be used to identify TUF1 receptor(s).

1. An isolated TUF1 polypeptide comprising an amino acid sequence having at least 83% sequence similarity to the amino acid sequence depicted in SEQ ID NO:1.

2. The isolated TUF1 polypeptide of claim 1 wherein the TUF1 polypeptide comprises an amino acid sequence having at least 83% sequence identity to the amino acid sequence depicted in SEQ ID NO:1.

3-35. (canceled)

36. A composition comprising the isolated polypeptide of claim 1 and a pharmaceutically acceptable carrier.

37. An isolated TUF1 polypeptide comprising an amino acid sequence having at least 83% sequence similarity to the amino acid sequence depicted in SEQ ID NO:2.

38. The isolated TUF 1 polypeptide of claim 37 wherein the TUF1 polypeptide comprises an amino acid sequence having at least 83% sequence identity to the amino acid sequence depicted in SEQ ID NO:2.

39. A composition comprising the isolated polypeptide of claim 37 and a pharmaceutically acceptable carrier.

40. An isolated polynucleotide that encodes a TUF1 polypeptide, wherein the TUF1 polypeptide comprises an amino acid sequence having at least 83% sequence similarity to the amino acid sequence depicted in SEQ ID NO:1.

41. The isolated polynucleotide of claim 40 wherein the TUF1 polypeptide comprises an amino acid sequence having at least 83% sequence identity to the amino acid sequence depicted in SEQ ID NO:1.

42. A composition comprising the isolated polynucleotide of claim 40 and a pharmaceutically acceptable carrier.

43. An isolated polynucleotide that encodes a TUF1 polypeptide, wherein the TUF1 polypeptide comprises an amino acid sequence having at least 83% sequence similarity to the amino acid sequence depicted in SEQ ID NO:2.

44. The isolated polynucleotide of claim 43 wherein the TUF1 polypeptide comprises an amino acid sequence having at least 83% sequence identity to the amino acid sequence depicted in SEQ ID NO:2.

45. A composition comprising the isolated polynucleotide of claim 43 and a pharmaceutically acceptable carrier.

46. An antibody composition that specifically binds to at least a portion of a TUF1 polypeptide, wherein the antibody specifically binds to at least a portion of the amino acid sequence depicted in SEQ ID NO:1, SEQ ID NO:2, SEQ ID NO:3, or SEQ ID NO:4.

47. The antibody composition of claim 46 wherein the antibody specifically binds to at least a portion of amino acids 6-20 of SEQ ID NO:3.

48. The antibody composition of claim 46 comprising polyclonal antibody.

49. The antibody composition of claim 46 comprising a monoclonal antibody.

50. The antibody composition of claim 46 comprising a pharmaceutically acceptable carrier.

51. A method comprising:
providing a composition comprising a TUF1 polypeptide, wherein the composition is effective to ameliorate at least one symptom or clinical sign of a condition treatable with a neurotrophin when the composition is administered to a subject in need of treatment for a condition treatable with a neurotrophin.

52. The method of claim 51 wherein the condition comprises brain injury.

53. The method of claim 51 wherein the condition comprises a fear disorder or anxiety disorder.

54. The method of claim 51 wherein the condition comprises male fertility.

55. The method of claim 51 wherein the composition comprises a TUF1 polypeptide comprising an amino acid sequence having at least 83% sequence similarity to the amino acid sequence depicted in SEQ ID NO:1.

56. The method of claim 51 wherein the composition comprises a TUF1 polypeptide comprising an amino acid sequence having at least 83% sequence similarity to the amino acid sequence depicted in SEQ ID NO:2.

57. A method comprising:
detecting expression of TUF1 in a tissue of a subject, wherein detecting TUF1 expression in the tissue indicates that the subject is at least at risk for developing a condition.

58. The method of claim 57 wherein the condition comprises hypertension.

59. The method of claim 57 wherein the condition comprises Huntington's Disease.

60. The method of claim 57 wherein the condition comprises Alzheimer's Disease.

61. The method of claim 57 wherein the condition comprises Parkinson's Disease.

62. A method comprising:
providing a composition comprising a TUF1 inhibitor, wherein administering an effective amount of the composition to a subject inhibits at least one activity of TUF1 in the subject.

63. The method of claim 62 wherein the composition comprises an isolated polynucleotide that encodes a TUF1 polypeptide, wherein the TUF1 polypeptide comprises an amino acid sequence having at least 83% sequence similarity to the amino acid sequence depicted in SEQ ID NO:1.

64. The method of claim 63 wherein the isolated polynucleotide comprises a siRNA that targets at least a portion of a polynucleotide that encodes TUF1 or an RNAi that targets at least a portion of a polynucleotide that encodes TUF1.

65. The method of claim 62 wherein the composition comprises an isolated polynucleotide that encodes a TUF1 polypeptide, wherein the TUF1 polypeptide comprises an amino acid sequence having at least 83% sequence similarity to the amino acid sequence depicted in SEQ ID NO:2.

66. The method of claim 65 wherein the isolated polynucleotide comprises a siRNA that targets at least a portion of a polynucleotide that encodes TUF1 or an RNAi that targets at least a portion of a polynucleotide that encodes TUF1.

67. The method of claim 62 wherein the composition comprises an antibody composition.

68. The method of claim 67 wherein the antibody composition comprises antibody that specifically binds to at least a portion of the amino acid sequence depicted in SEQ ID NO:1, at least a portion of the amino acid sequence depicted in SEQ ID NO:2, at least a portion of the amino acid sequence depicted in SEQ ID NO:3, or at least a portion of the amino acid sequence depicted in SEQ ID NO:4.

69. The method of claim 68 wherein the antibody specifically binds to at least a portion of amino acids 6-20 of SEQ ID NO:3.

70. The method of claim 67 wherein the antibody composition comprises polyclonal antibody.

71. The method of claim 67 wherein the antibody composition comprises a monoclonal antibody.

72. The method of claim 62 wherein the condition comprises hypertension.

73. The method of claim 62 wherein the condition comprises obesity.
